SEQUENCE LISTING (14 sequences)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - DNA, 2229 bp, Homo sapiens
atggatgcaatgaagagagggctctgctgtgtgctgctgctgtgtggagcagtcttcgtttcgcccagccaggaaatccatgcccgattcagaagaggcgccagaaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaa

SEQ ID NO: 3 - protein, 742 aa, Homo sapiens
MDAMKRGLCCVLLLCGAVFVSPSQEIHARFRRGARKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 4 - protein, 696 aa, Homo sapiens
KHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 5 - protein, 11 aa, Homo sapiens
KSSNEATNITP

SEQ ID NO: 6 - protein, 23 aa, Homo sapiens
MDAMKRGLCCVLLLCGAVFVSPS

SEQ ID NO: 7 - protein, 12 aa, Homo sapiens
QEIHARFRRGAR

SEQ ID NO: 8 - protein, 61 aa, Homo sapiens
KHNMKAFLDELKAENIKKFLYNFTQIPHLAGTEQNFQLAKQIQSQWKEFGLDSVELAHYDV

SEQ ID NO: 9 - protein, 237 aa, Homo sapiens
LLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEV

SEQ ID NO: 10 - protein, 247 aa, Homo sapiens
TRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRD

SEQ ID NO: 11 - protein, 151 aa, Homo sapiens
YAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 12 - protein, 10 aa, Homo sapiens
KSNPIVLRMM

SEQ ID NO: 13 - DNA, 12 bp, Homo sapiens
tgcccaccgtgc

SEQ ID NO: 14 - DNA, 2229 bp, Homo sapiens
ttaggctacttcactcaaagtctctgcagctgcctgcactgtgaaggctgcaacataaatctgtctcttcacttctccccaggccttggaagggtccactttgctttcaatatcaaacagagcatcataaattcctgggaatgactcccctgcatacttgttgtggctgcttggagcatagatgacatgcctataaaaaggcctgtctggtaaccctaatggatcaataaatgctctttccagaaacatgagttgatcattcatcattcttaatactattgggttgcttttgtcaaagtcctggagtctctcactgaacttggaagcaatttctgtaaaattctttactgcagaaaaaagtgaatcaaatgatacactgtatgtcttcatttcctgtggatgtttcatagaaatactgtagattttgtcagcatactttcttaaaactacagcataatctcgacaatcaaaagggagcactatggaattggctagctcaaacaccatccctcctcgaacctgggccacagtgaggtgatatttaaacattggatcataaaacttttccaccaactcatatgtttcatagacactgtgatacagtggatagccgctgaatttgtttgtttcccaatttttagtataccgtgctctgcctgaagcaattccaagtcgttggaagaacacctcaaaatcatttccagatcccaatttgcttatcctgggcatgccactgaactctggggaaggactttttttagtccaactttcataaagagatttgccttcaaagccttcatcagggcttttcagctcttttgttaggttgtgtaccaagctgtacatcagcggtgtacaatcaactctcagagtgtagtttccttctatagatgagtcagcattaatataagccacgccacgctcttgaaggagtcttgaattctcctctgcccactcagtagaaccaagaagaccaaattcttctgcatcccagcttgcaaacaaaattgttcttctaggtctccacccttcctttttcagtgttccaaagctcctcacaatttcatgaacaacagctgctccactctgagggtcaataccaccaaacacccatgagtcccggtgacctcccagaatgacatatctgtctggttccactgctcctctgagagtacctatcacattgtaaattcttgtcacttcattggtagagtggatgtgcatcttgactttttgtgtagaaaagtttccagtaaagccaggtccaacattgtagggcactttgagacttcctctccagctgctatctggtggtgctgagccacccattttttctaggagcttctgtgcatcatagtatccaattggatgaacaggaatacttggaagaccaacagcctctgcaattccacgcctataagcatattcatttgctgggtaacctggtgtgagagggtctcctgcaccattcagatttaggatatttccacgctggacaccacctccaggaagattccaaccatctggataggacttcaccccaggagcaaagtagtcagcagggtcggagtagagaatgactcctttggcccctgccagctgggcatttttaaccttatttcctctgaaaactttcccatatctggcaattacaattttcccagagcaattgattttcatgtcccgttccaatttaaagaagtcttcagttcgtgcatagttaacatacactagatcgccctctggcattccttgaggagagaaagcactgaaaggtggtacaatatccgaaacattttcatatcctggaggaggtggttcaaataatgatgtgttgaaaatctcatttccatcttcattaattattgagatgtagttgggatgagtcttatttgggtaggacaacaggacatcataatgtgctagctcaacagaatccaggccaaattctttccactgggattgaatttgctttgcaagctgaaagttttgttctgttcctgctaaatgtggtatctgtgtaaaattatataagaacttcttgatgttctcagctttcaattcatccaaaaatgctttcatattatgctttggagtaatgttagtagcttcattggaggattttctggcgcctcttctgaatcgggcatggatttcctggctgggcgaaacgaagactgctccacacagcagcagcacacagcagagccctctcttcattgcatccat

(19) United States
(12) Patent Application Publication
(10) Pub. No.: US 2009/0311225 A1
(43) Pub. Date: Dec. 17, 2009

(54) Compositions of and Methods of Using Stabilized PSMA Dimers

(75) Inventor: Kanaka Raju Koduri, White Plains, NY (US)

Correspondence Address: WOLF GREENFIELD & SACKS, P.C., 600 ATLANTIC AVENUE, BOSTON, MA, 02210-2206, US

(73) Assignee: PSMA Development Company, LLC, Tarrytown, NY (US)

(21) Appl. No.: 12/085,040

(22) PCT Filed: Nov. 14, 2006
(86) PCT No.: PCT/US2006/044298

§ 371 Date: Jul. 6, 2009

Related U.S. Application Data
(60) Provisional application No. 60/736,935, filed on Nov. 14, 2005.

(51) Int. Cl. A61K 38/17; C07K 14/47; C12N 15/12; C12N 15/85; C12N 5/10; A61K 31/7088; A61K 35/12; A61K 48/00; C12P 21/00; A61P 35/00; C12P 21/02

(52) U.S. Cl. 424/93.21; 530/350; 514/12; 536/23.5; 435/320.1; 435/325; 514/44 R; 424/93.2; 435/69.6; 435/69.1

(57) ABSTRACT

The invention includes cysteine-modified PSMA polypeptides and disulfide-bond-stabilized dimers thereof, compositions and kits containing the cysteine-modified PSMA polypeptides, including dimers thereof, as well as methods of producing and using these compositions. Such methods include methods for eliciting or enhancing an immune response to cells expressing PSMA, methods of producing antibodies to PSMA, including dimeric PSMA, as well as methods of treating cancer, such as prostate cancer.

### RELATED APPLICATIONS

This application claims the benefit under 35 U.S.C. § 119 of U.S. provisional application 60/736,935, filed Nov. 14, 2005, the contents of which are incorporated herein by reference in their entirety.

### FIELD OF THE INVENTION

This invention relates generally to the field of cancer-associated polypeptides, compositions of and kits including these polypeptides, as well as methods of their production and use. More specifically, the invention relates, in part, to compositions of cysteine-modified PSMA polypeptides, in particular cysteine-modified PSMA polypeptides that form disulfide-bond-stabilized PSMA dimers, and methods of their production and use.

### BACKGROUND OF THE INVENTION

Prostate cancer is the most common malignancy and the second leading cause of cancer death in men in the United States. Localized prostate cancer typically is treated with surgery or radiation, and recurrent disease can be controlled temporarily with androgen ablation. However, almost all prostate carcinomas eventually become hormone-refractory and then rapidly progress. Hormone-refractory or androgen-independent prostate cancer has proven to be largely resistant to conventional chemotherapy. With the exception of palliative care, the only approved chemotherapy is docetaxel in combination with prednisone, which offers a modest (2.4 month) survival benefit. New molecularly targeted therapies are needed.

### SUMMARY OF THE INVENTION

The present invention relates, in part, to cysteine-modified PSMA polypeptides, compositions and kits containing cysteine-modified PSMA polypeptides as well as methods of producing and using these compositions. In some embodiments the cysteine-modified PSMA polypeptides are cysteine-modified PSMA polypeptides that form disulfide-bond-stabilized PSMA dimers. Compositions of and methods of using the disulfide-bond-stabilized PSMA dimers are also provided.

In one aspect of the invention a cysteine-modified PSMA polypeptide is provided which comprises a cysteine-modified stalk region, and an amino acid sequence set forth as SEQ ID NO: 4 or a fragment thereof. The amino acid sequence of SEQ ID NO: 4 corresponds to residues 55-750 of full-length PSMA (the amino acid sequence of full-length PSMA is set forth in SEQ ID NO: 1). In one embodiment the cysteine-modified PSMA polypeptide consists of a cysteine-modified stalk region and the amino acid sequence set forth as SEQ ID NO: 4.

In another embodiment the cysteine-modified stalk region has an amino acid sequence as set forth in SEQ ID NO: 5 except that one or more residues of SEQ ID NO: 5 are substituted with cysteine. In another embodiment one, two or three residues of SEQ ID NO: 5 are substituted with cysteine. In still another embodiment one of the residues substituted with cysteine corresponds to the residue at position 1, 2, 3, 4, 5, 6 or 7 of SEQ ID NO: 5. In yet another embodiment one of the residues substituted with cysteine corresponds to the residue at position 1, 2, 3, 4 or 5 of SEQ ID NO: 5. In a further embodiment one of the residues substituted with cysteine corresponds to the residue at position 1, 2 or 3 of SEQ ID NO: 5. In still a further embodiment one of the residues substituted with cysteine corresponds to the residue at position 3 of SEQ ID NO: 5. In another embodiment one residue of SEQ ID NO: 5 is substituted with cysteine.

In yet another embodiment the cysteine-modified stalk region has an amino acid sequence as set forth in SEQ ID NO: 5 except that one of the residues at position 2, 3, 4, 5, 6, 7, 8, 9, 10 or 11 of SEQ ID NO: 5 is substituted with cysteine and the residue at position 1 of SEQ ID NO: 5 is substituted with a non-positively charged amino acid. In one embodiment the non-positively charged amino acid is cysteine, glycine, alanine, glutamine, glutamic acid, aspartic acid or asparagine.

In still a further embodiment the cysteine-modified stalk region has the amino acid sequence as set forth in SEQ ID NO: 5 except that one or more cysteine residues are inserted therein. In one embodiment the one or more cysteine residues are inserted after the residue that corresponds to the residue at position 1 of SEQ ID NO: 5. In another embodiment two cysteine residues are inserted after the residue that corresponds to the residue at position 1 of SEQ ID NO: 5. In one embodiment the cysteine residues are inserted contiguously. In another embodiment the cysteine residues are inserted non-contiguously.

In a further embodiment the one or more cysteine residues that are inserted are part of an amino acid sequence, X¹ₙ-X²-X³-X⁴-X⁵-X⁶ₙ. In one embodiment n is 0 or 1. In some of these embodiments X¹, X², X³, X⁴, X⁵, X⁶ each can be any amino acid residue provided that the inserted amino acid sequence contains at least one cysteine residue. In another embodiment the amino acid sequence contains at least two, three or four cysteines. In still another embodiment the one or more cysteine residues that are inserted are part of the amino acid sequence, C-X¹-X²ₙ-C, wherein X¹ and X² are each any amino acid residue and n is 0, 1 or 2. In one embodiment n is 1. In another embodiment X¹ and X² are each proline or serine. In a further embodiment X¹ and X² are each proline. In yet another embodiment X¹ is proline and X² is serine.

In another embodiment the cysteine-modified stalk region has the amino acid sequence as set forth in SEQ ID NO: 5 except that the residue at position 3, 5, 6 or 7 of SEQ ID NO: 5 is substituted with a cysteine.

In yet another embodiment the cysteine-modified stalk region has the amino acid sequence as set forth in SEQ ID NO: 5 except that the amino acid sequence encoded by SEQ ID NO: 13 or a degenerate thereof is inserted therein or at the amino or carboxy terminus. In one embodiment the sequence encoded by SEQ ID NO: 13 or a degenerate thereof is inserted between the residues at positions 1 and 2 of SEQ ID NO: 5.

In another aspect of the invention compositions are provided comprising one or more of the cysteine-modified PSMA polypeptides described herein. In still another aspect of the invention compositions are provided comprising a disulfide-bond-stabilized PSMA dimer, which is formed from two of the cysteine-modified PSMA polypeptides provided herein.

In yet another aspect of the invention nucleic acid molecules are provided that encode a cysteine-modified PSMA polypeptide. In one embodiment the nucleic acid is DNA or RNA.

In still another aspect of the invention vectors comprising a nucleic acid molecule encoding a cysteine-modified PSMA polypeptide are provided. In one embodiment the nucleic acid molecule encoding a cysteine-modified PSMA polypeptide is operably linked to a promoter. In another embodiment the vector is a plasmid or viral vector. In still another embodiment the vector is a DNA plasmid. In a further embodiment the viral vector is a pox virus, a herpes virus, adenovirus, vaccinia virus or alphavirus vector.

In a further aspect of the invention host cells transformed or transfected with a vector as described herein are provided.

In yet another aspect of the invention compositions comprising cysteine-modified PSMA polypeptides, including dimers thereof, are provided. In a further aspect of the invention compositions comprising a nucleic acid encoding a cysteine-modified PSMA polypeptide are provided. In still a further aspect of the invention compositions comprising a vector or host cell as described herein are provided. In one embodiment these compositions are therapeutic compositions. In another embodiment these compositions are vaccine compositions.

In one embodiment the compositions provided further comprise an adjuvant. In another embodiment the adjuvant is alum; monophosphoryl lipid A; a saponin; QS-7; QS-17; QS-18; QS-21; a saponin fraction; a saponin-based adjuvant; SaponImmune™; PolysaccImmune™; SynthImmune™; an immunostimulatory oligonucleotide; incomplete Freund's adjuvant; complete Freund's adjuvant; montanide; MONTANIDE ISA51; MONTANIDE ISA720; vitamin E, a water-in-oil emulsions prepared from a biodegradable oil; Quil A; a micellular mixture of Quil A and cholesterol known as immunostimulating complexes (ISCOMS); a MPL and mycobacterial cell wall skeleton combination; ENHANZYN™; RC-529; RC-552; CRL-1005, L-121, alpha-galactosylceramide; a composition of biodegradable particles composed of poly-lactide-co-glycolide (PLG); a composition of aluminum or iron oxide beads or a combination thereof. In another embodiment the adjuvant is alum or a saponin-based adjuvant. In one embodiment the saponin-based adjuvant is QS-21.

In another embodiment the compositions provided further comprise an additional therapeutic agent. In one embodiment the therapeutic agent is docetaxel. In another embodiment the therapeutic agent is prednisone. In a further embodiment the compositions provided further comprise a combination of docetaxel and prednisone.

In still another embodiment the compositions provided further comprise a cytokine.

In yet another embodiment the compositions provided further comprise a pharmaceutically acceptable carrier. In a further embodiment the compositions provided are sterile. In another embodiment the compositions provided are physiologically acceptable. In still another embodiment the compositions provided are in a liquid or lyophilized form.

In another aspect of the invention a method of stimulating an immune response by administering a composition as provided herein to a subject in an amount effective to stimulate an immune response is provided. In one embodiment the composition comprises a cysteine-modified PSMA polypeptide in monomeric or dimeric form. In another embodiment the composition comprises a nucleic acid molecule that encodes a cysteine-modified PSMA polypeptide. In yet another embodiment the composition comprises a vector or host cell as provided herein. In a further embodiment the composition comprises or further comprises a full-length PSMA polypeptide or a fragment thereof, native PSMA dimer, or a nucleic acid encoding the full-length PSMA polypeptide or fragment thereof. In another embodiment the composition comprises or further comprises rsPSMA, such as rsPSMA in its dimeric form. In a further embodiment the composition comprises or further comprises a nucleic acid that encodes rsPSMA.

In still another embodiment the method further comprises administering one or more booster doses of a composition provided herein. In one embodiment the booster dose composition comprises a cysteine-modified PSMA polypeptide in monomeric or dimeric form. In another embodiment the booster dose composition comprises a nucleic acid molecule that encodes a cysteine-modified PSMA polypeptide. In yet another embodiment the booster dose composition comprises a vector or host cell as provided herein. In yet another embodiment the booster dose composition comprises a full-length PSMA polypeptide or a fragment thereof, native PSMA dimer, or a nucleic acid encoding the full-length PSMA polypeptide or fragment thereof. In another embodiment the booster dose composition comprises rsPSMA, such as rsPSMA in its dimeric form. In a further embodiment the booster dose composition comprises a nucleic acid that encodes rsPSMA. In another embodiment the immune response is an immune response to cells in the subject that express PSMA. In one embodiment the cells that express PSMA are cancer cells.

In another embodiment the cells that express PSMA are prostate cancer cells. In another embodiment the subject has or has been treated for cancer. In still another embodiment the subject has or has been treated for prostate cancer.

In a further embodiment the composition (initial or booster dose composition) is administered by intravenous, intramuscular, subcutaneous, parenteral, spinal, intradermal or epidermal administration. In one embodiment the composition is administered by subcutaneous or intramuscular administration.

In still a further embodiment the method further comprises harvesting antibodies produced as a result of the immune response.

In yet another aspect of the invention a method of treating cancer in a subject by administering to the subject a therapeutically effective amount of a composition described herein, wherein the composition is effective in treating cancer, is provided. In one embodiment the cancer is prostate cancer. In another embodiment the method further comprises administering to the subject a conventional prostate cancer therapy. In one embodiment the conventional prostate cancer therapy is surgery, radiation, cryosurgery, thermotherapy, hormone therapy or chemotherapy. In still another embodiment the method further comprises administering to the subject docetaxel, prednisone or both.

In another aspect of the invention a method of producing a PSMA polypeptide by modifying a nucleic acid molecule that encodes a PSMA polypeptide comprising the stalk region of PSMA so that the nucleic acid molecule codes for a cysteine residue within the stalk region, and transfecting or transforming cells with a vector containing the modified nucleic acid molecule is provided. In one embodiment the nucleic acid molecule is modified to code for a cysteine substitution within the stalk region. In another embodiment the nucleic acid molecule is modified to code for a cysteine insertion within the stalk region. In still another embodiment the method further comprises harvesting and purifying PSMA polypeptide expressed by the transfected or transformed cells. In one embodiment the PSMA polypeptide expressed is in a disulfide-bonded dimeric form.

In still another aspect of the invention a method of producing a PSMA polypeptide by transfecting or transforming cells with a vector encoding the PSMA polypeptide, and contacting the cells with media comprising an anti-apoptotic agent, polyethylene glycol (PEG) or both is provided. In one embodiment the anti-apoptotic agent is dextran sulfate, tropolone, a caspase inhibitor or the BCL2 gene product. In another embodiment the anti-apoptotic agent is dextran sulfate. In yet another embodiment the caspase inhibitor is Z-VAD, AEVD-FMK, LEED-FMK or Z-DEVD-FMK. In a further embodiment the PEG has a molecular weight of 2000, 3000, 4000, 6000 or 8000. In one embodiment the PEG is PEG 8000. In still another embodiment the PSMA polypeptide has a cysteine-modification. In a further embodiment the method further comprises harvesting and purifying PSMA polypeptide expressed by the transfected or transformed cells. In another embodiment PSMA polypeptide expressed by the transfected or transformed cells is in a disulfide-bonded dimeric form.

In another aspect of the invention a PSMA polypeptide, or dimer thereof, or composition comprising the PSMA polypeptide or dimer thereof produced by the methods described herein is also provided.

In a further aspect of the invention a kit which comprises a composition described herein and instructions for use is provided.

In another aspect of the invention a kit which comprises a composition described herein, an adjuvant and instructions for mixing is provided.

In still another aspect of the invention a kit which comprises a composition described herein, a diluent and instructions for mixing is provided.

In one embodiment of some of the aspects of the invention the composition is provided in a vial or ampoule with a septum or a syringe. In another embodiment the composition is in a liquid or lyophilized form.

Each of the limitations of the invention can encompass various embodiments of the invention. It, therefore, is anticipated that each of the limitations of the invention involving any one element or combinations of elements can be included in each aspect of the invention. These and other aspects of the invention will be described in further detail in connection with the detailed description of the invention.

### BRIEF DESCRIPTION OF THE DRAWINGS

FIG. 1 shows that Lonza pEE14.4\rsPSMA obtained from a plasmid miniprep resulted in the appropriate 1.3 kb and 0.8 kb bands according to the location of EcoR1 and HindIII restriction sites.

FIG. 2 shows that Lonza pEE14.4\rsPSMA samples with the amino acid insertion obtained from a plasmid miniprep resulted in the appropriate 1.3 kb and 0.8 kb bands according to the location of EcoR1 and HindIII restriction sites.

FIG. 3 illustrates that the desired PCR band to confirm the presence of the insertion mutation is approximately 300 base pairs in length, the distance between the PCR diagnostic primer and the reverse primer used. 1 kb size markers are shown in lanes 4 and 12. Samples in lanes 1, 2, 3, 6, 9, 11, 13, 14 and 15 show the PCR band of desired length, indicating that those DNA samples contain the desired mutations. Samples in lanes 1, 2 and 3 most clearly demonstrate the desired band.

FIG. 4 provides results whereby lanes 1-6 are samples from the 389E-C PCR diagnostic reaction. Lane 7 is a 1 kb size marker. Lanes 8-13 are samples from the 623P-C PCR diagnostic reaction. A 700 bp non-specific reaction is visible in the 389E-C samples. However, there is a clear 850 bp PCR band of the desired length present in samples of lanes 2-6 which is not present in the sample in lane 1. With regard to the 623P-C samples, lanes 9, 11, 12 and 13 exhibit the desired 250 bp PCR band, while lanes 8 and 10 do not.

FIG. 5 illustrates that under denaturing, non-reducing conditions wild type (wt) rsPSMA is seen almost completely in monomer form, while rsPSMA containing the engineered insertion in the stalk region is present mainly as a dimer. The monomer and dimer bands shown are of the expected molecular weight, and purified rsPSMA protein standard behaved as predicted falling apart into monomer configuration under denaturing conditions. Monomer and dimer bands of the expressed mutant ran at the same molecular weight as the purified rsPSMA protein standard.

FIG. 6 provides results from a dot blot of transiently expressed wt rsPSMA and insertion mutant probed with a human monoclonal anti-PSMA antibody (anti-PSMA hmAb 006) which recognizes the dimeric form of PSMA. The blot demonstrates that the insertion mutant was as reactive to anti-PSMA hmAb 006 as wt rsPSMA.

FIG. 7 provides the results from a Western blot of transiently expressed rsPSMA with a four amino acid insertion in the stalk region immunoprecipitated using a human monoclonal anti-PSMA antibody (anti-PSMA hmAb 006). This mutant protein selected with anti-PSMA hmAb 006 appears entirely in dimer configuration under denaturing conditions.

FIG. 8 provides the results from a reduced Western blot, which illustrates the difference between the amount of protein expressed in cells which were in expression media containing dextran sulfate and cells in expression media not containing dextran sulfate. Dextran sulfate has been found to enhance the transient expression of rsPSMA and the rsPSMA insertion mutant.

FIG. 9 shows that while dextran sulfate improves the overall expression of insertion mutant #1, the introduction of PEG into the expression media seems to increase the dimer to monomer ratio of insertion mutant #1.

FIG. 10 provides the structure of human transferrin receptor (hTfR) with the stalk region.

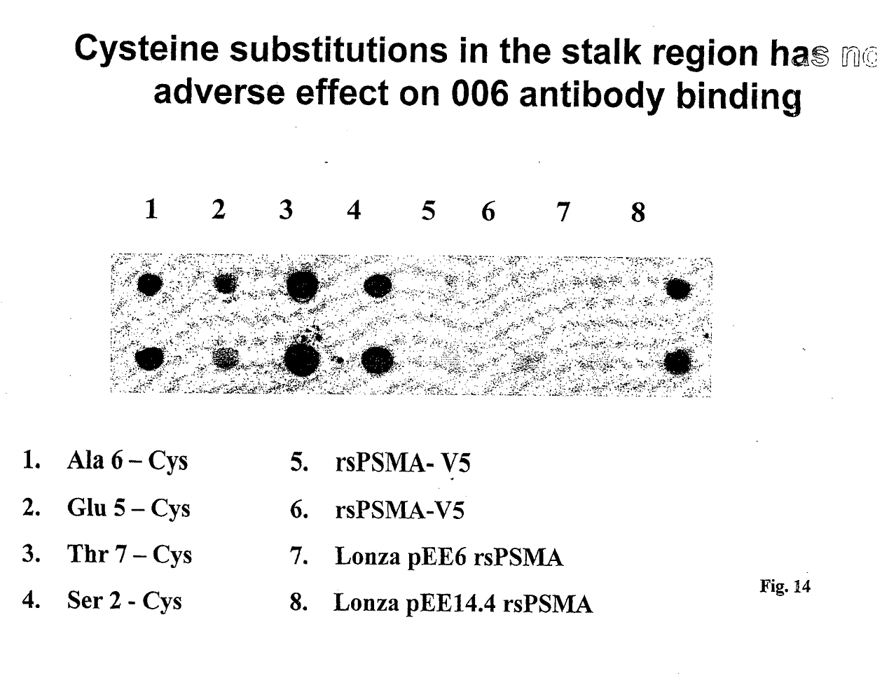

FIG. 11 illustrates the organization of rsPSMA.

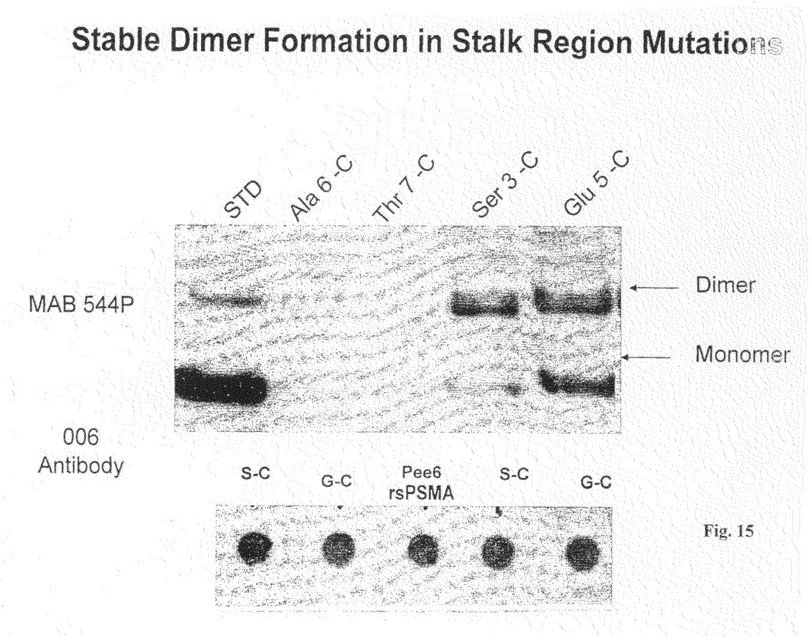

FIG. 12 shows some cysteine mutations of the stalk region (domain III) and the helical region of rsPSMA.

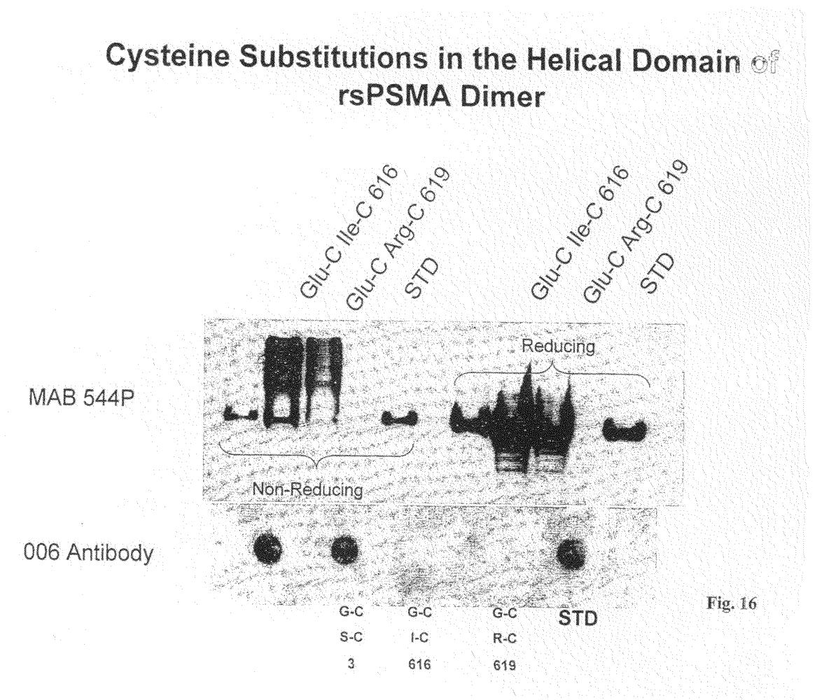

FIG. 13 provides the results of a dot blot assay which shows that transiently expressed rsPSMA is recognized by anti-PSMA hmAb 006.

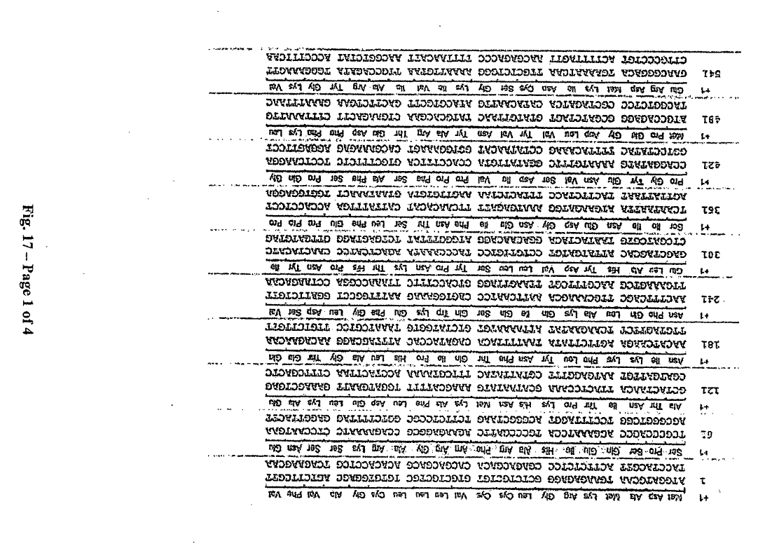

FIG. 14 illustrates that cysteine substitutions in the stalk region has no adverse effect on anti-PSMA hmAb 006 binding.

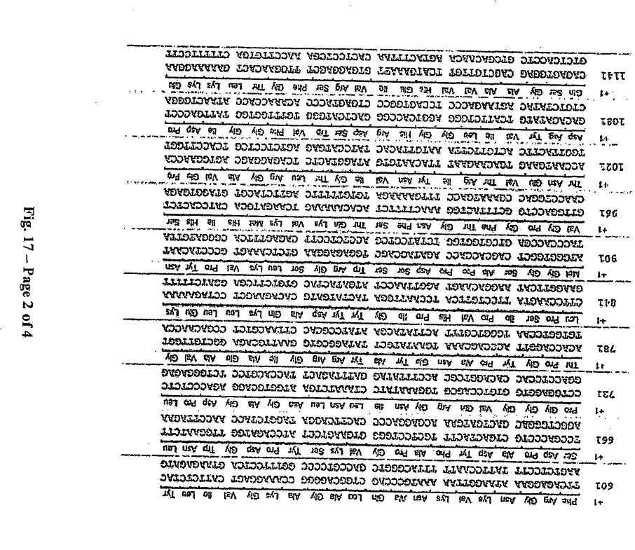

FIG. 15 shows stable dimer formation of stalk region mutants.

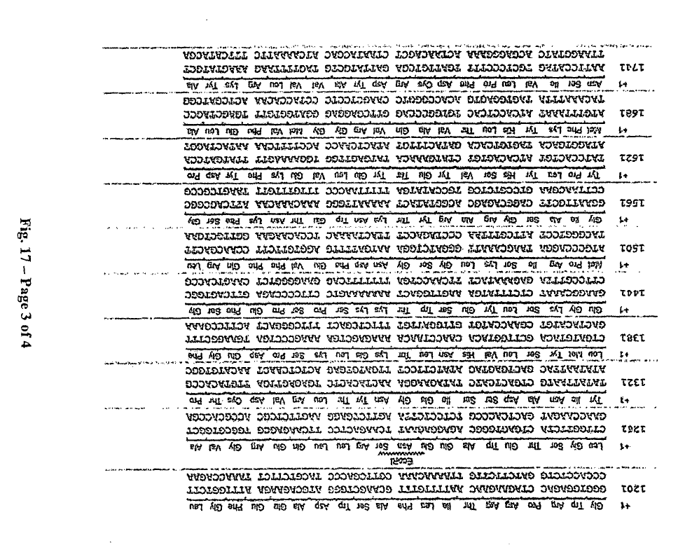

FIG. 16 illustrates the results of cysteine substitutions in the helical domain of rsPSMA dimer.

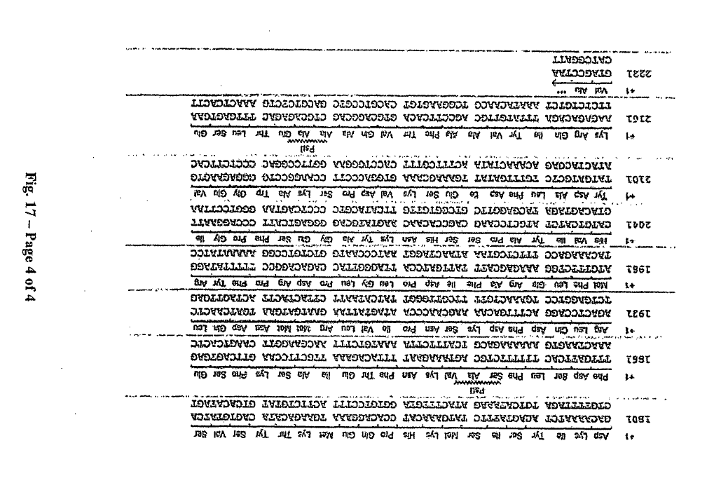

FIG. 17 provides the amino acid (SEQ ID NO: 3) and nucleic acid sequence (SEQ ID NO: 2) of rsPSMA with tPA signal sequence and tPA pro-sequence. The complement of SEQ ID NO:2 (5′ to 3′) is provided as SEQ ID NO: 14.

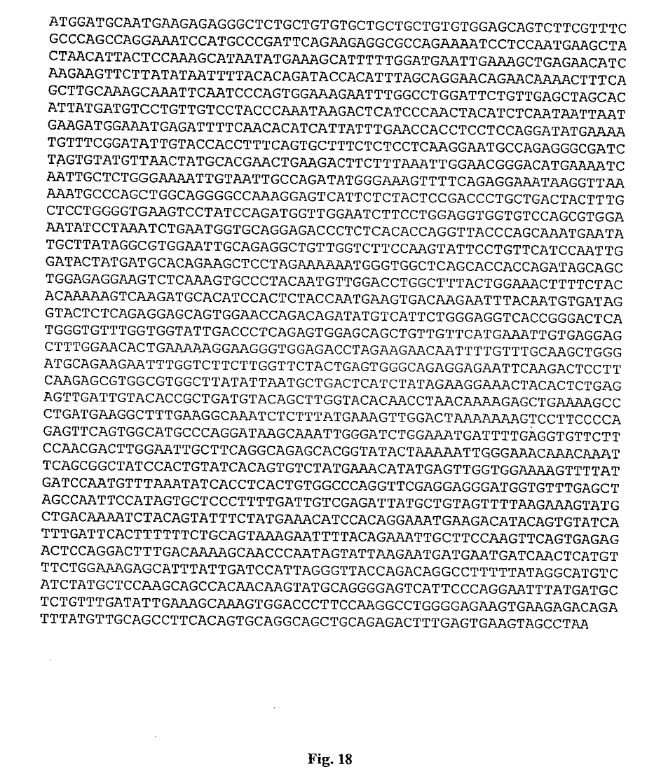

FIG. 18 provides the rsPSMA coding region with tPA signal sequence and tPA pro-sequence (SEQ ID NO: 2).

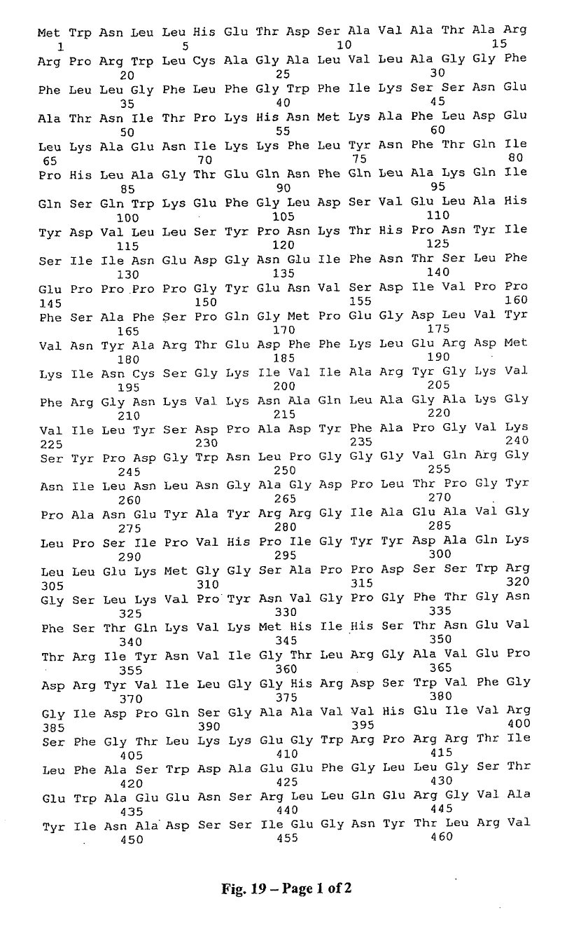

FIG. 19 provides the amino acid sequence of full-length PSMA (SEQ ID NO: 1).

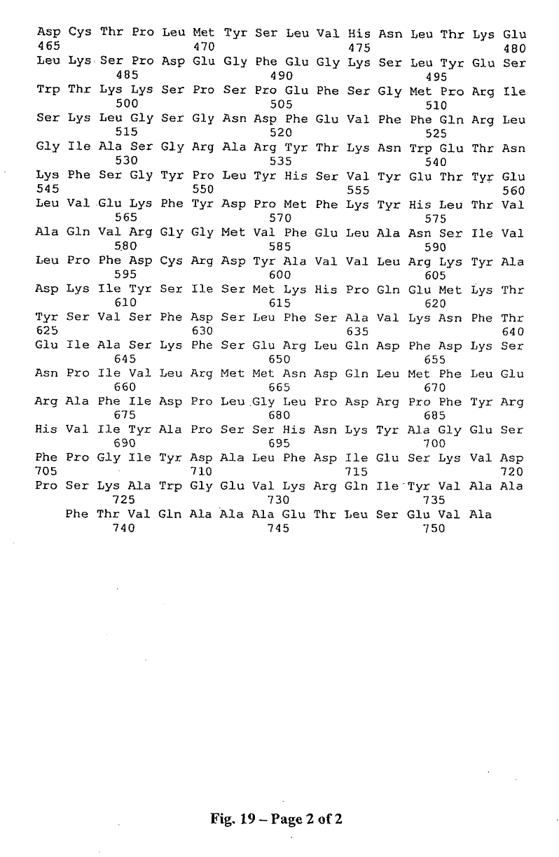

### DETAILED DESCRIPTION OF THE INVENTION

Prostate specific membrane antigen (PSMA) is a 100 kD type II membrane glycoprotein expressed in prostate tissues and was originally identified by reactivity with a monoclonal antibody, designated 7E11-CS (Horoszewicz et al., 1987, Anticancer Res. 7:927-935; U.S. Pat. No. 5,162,504). PSMA was characterized as a type II transmembrane protein having a sequence with some homology with the transferrin receptor (Israeli et al., 1994, Cancer Res. 54:1807-1811) and with NAALADase activity (Carter et al., 1996, Proc. Natl. Acad. Sci. U.S.A. 93:749-753). More importantly, PSMA is expressed in increased amounts in prostate cancer, and elevated levels of PSMA are also detectable in the sera of these patients (Horoszewicz et al., 1987; Rochon et al., 1994, Prostate 25:219-223; Murphy et al., 1995, Prostate 26:164-168; and Murphy et al., 1995, Anticancer Res. 15:1473-1479). PSMA expression increases with disease progression, becoming highest in metastatic, hormone-refractory disease for which there is no present therapy. Data also indicate that PSMA is also abundantly expressed on the neovasculature of other important cancers/tumors, including, for example, cancerous tissue of metastatic bone marrow and cancerous tissue of metastatic lymph nodes as well as breast, bladder, urothelial, pancreatic, sarcoma, melanoma, lung, liver, colon, rectal and kidney cancer/tumor cells, but not on normal vasculature.

Prostate-specific membrane antigen (PSMA) polypeptides and the nucleic acids that encode them can serve as vaccines for cancer. PSMA in its native form is a homodimer. PSMA is expressed on tumor cells as a noncovalent homodimer. A truncated PSMA protein, lacking the transmembrane and cytoplasmic domains, also forms noncovalent homodimers (rsPSMA, amino acids 44-750 of full-length PSMA (SEQ ID NO: 1)) (PCT Publication WO 03/34903; Schülke, N. et al. (2003) PNAS, 100, 12590-12595), and the rsPSMA dimers but not monomers display a native conformation. Additionally, when used as a protein vaccine to immunize animals, rsPSMA dimers elicited antibodies that efficiently recognized PSMA-expressing tumor cells. Formulations have been designed to preserve/enhance the dimeric structure of rsPSMA in solutions (U.S. Patent Publication US 2005/0215472 A1).

No native cysteine-mediated covalent bond exists between the monomer polypeptides of PSMA. As described herein, disulfide-bonded rsPSMA dimers were engineered using cysteine substitutions and cysteine insertions at various locations to form covalently linked, stable dimers. The sites for engineering disulfide-bond-forming cysteine substitutions and cysteine insertions in rsPSMA were selected by direct observation of the crystal structure of the PSMA dimer and by observing the crystal structure of the helical domain of a related protein, human transferrin receptor (hTfR), reported to facilitate dimerization (Lawrence et al., Science, Vol. 286, pp. 779-782, 1999.) Lawrence et al. (Science, Vol. 286, pp. 779-782, 1999) also reported a region of 35 amino acid sequence between the transmembrane domain and the beginning of the protease-like domain of hTfR, termed the stalk. The stalk region of the hTfR contains two cysteines which were reported as not required for dimerization of hTfR. With an alignment of rsPSMA with hTfR (Lawrence et al., www.sciencemag.org/feature/data/1043272.shl) an 11 amino acid corresponding stalk region, which does not contain cysteines, is observed. The stalk region of rsPSMA was also selected as a site for engineering disulfide-bond-forming cysteine substitutions and cysteine insertions to form stable, covalently linked rsPSMA homodimers.

It was surprising that cysteine substitutions in the helical domain of rsPSMA resulted in insoluble protein. The engineering of cysteines in the stalk region of rsPSMA polypeptides, however, led to the formation of stable rsPSMA dimers with the native conformation of rsPSMA retained. This result was also surprising in light of the report by Lawrence et al. (Science, Vol. 286, pp. 779-782, 1999) that the cysteine-containing stalk region of hTfR is not required for dimer formation.

The present invention provides, in part, cysteine-modified PSMA polypeptides, compositions and kits containing the cysteine-modified PSMA polypeptides as well as methods of producing and using these compositions. Such methods include methods for eliciting or enhancing an immune response to PSMA, such as native PSMA in dimer form, and/or cells expressing PSMA, such as cancer cells. Such methods also include methods of producing antibodies specific to PSMA, including dimeric PSMA and/or PSMA expressed on cells, such as cancer cells, as well as methods of treating cancer, such as prostate cancer. The cysteine-modified PSMA polypeptides of the invention include those that form disulfide-bond-stabilized PSMA dimers, and compositions of and methods of using these dimers are also provided.

The term “cysteine-modified PSMA polypeptide”, as used herein, is intended to refer to a PSMA polypeptide that comprises a cysteine modification (i.e., one or more cysteine substitutions, insertions or some combination thereof). In some embodiments the cysteine-modified PSMA polypeptide comprises a cysteine-modified stalk region and an amino acid sequence as set forth in SEQ ID NO: 4 or a fragment thereof. The amino acid sequence set forth as SEQ ID NO: 4 corresponds to residues 55-750 of full-length PSMA. The amino acid sequence of full-length PSMA is set forth in SEQ ID NO: 1. The cysteine-modified PSMA polypeptide, in some embodiments, forms a disulfide-bond-stabilized PSMA dimer, which has a conformation of a native dimer. When two cysteine-modified PSMA polypeptides form a disulfide-bond-stabilized PSMA dimer, disulfide bonds are formed between cysteine residues of the polypeptides such that the dimer contains at least one cistine. When a cysteine-modified PSMA polypeptide contains more than one cysteine residue, the cysteines of a cysteine-modified PSMA polypeptide preferably bond with cysteines of another cysteine-modified PSMA polypeptide. In other words, the disulfide bonds formed are preferably intermolecular and are not intramolecular. When in dimeric form the two cysteine-modified PSMA polypeptides, in some embodiments, have a conformation of native dimeric PSMA. The disulfide-bond-stabilized PSMA dimers provided can be used, in some embodiments, to generate antibodies that are specific for PSMA, native dimeric PSMA and/or PSMA-expressing cells. They can also be used, in some embodiments, to generate a specific cytotoxic T cell response and/or antibodies that elicit cytotoxic T cells.

As used herein, an antibody that is “specific for PSMA” refers to antibody binding to PSMA as its predetermined antigen. Typically, the antibody binds with an affinity that is at least two-fold greater than its affinity for binding to a non-specific antigen (e.g., BSA, casein). “Non-specific antigens” are antigens unrelated to PSMA.

The cysteine-modified PSMA polypeptides provided are, in some embodiments, capable of forming disulfide-bond-stabilized PSMA dimers. In some embodiments, the cysteine-modified PSMA polypeptides are those that are capable of forming a dimer like that of native PSMA. A “dimer like that of native PSMA” includes two PSMA polypeptides that have a conformation of the PSMA protein as it is found in nature and/or on PSMA-expressing cancer cells or a conformation which will result, when injected in an animal, in the generation of antibodies that recognize at least one antigenic epitope of the native PSMA dimer (i.e., associated in a way such as to form an antigenic region as found in the native PSMA dimer or one capable of generating cross-reacting antibodies to an antigenic region as found in the native PSMA dimer). Some of the antibodies generated to the cysteine-modified PSMA polypeptides, including dimers thereof, provided herein are, therefore, capable of specifically binding the native PSMA dimer. In some embodiments, such antibodies recognize native PSMA dimer but not PSMA monomer or have greater specificity for the native PSMA dimer rather than the monomer (i.e., is “specific for the native PSMA dimer”.) In one embodiment, therefore, the PSMA polypeptides provided can be used to generate antibodies that are specific for the native PSMA dimer (also referred to herein as native dimeric PSMA, dimeric form of native PSMA, etc.)

The cysteine-modified PSMA polypeptides provided, and disulfide-bond-stabilized PSMA dimers thereof, therefore, can, in some embodiments, be used to generate antibodies that specifically bind the cysteine-modified PSMA polypeptides or dimers thereof. In some embodiments, the antibodies generated also specifically bind native PSMA dimer and/or PSMA-expressing cells, such as PSMA-expressing cancer cells. The antibodies generated can also, in some embodiments, elicit cytotoxic T cells. In one embodiment the antibodies specifically bind a cysteine-modified PSMA polypeptide dimer, native PSMA dimer and PSMA-expressed on cancer cells. In another embodiment the antibodies specifically bind a cysteine-modified PSMA polypeptide dimer, native PSMA dimer, PSMA-expressed on cancer cells and elicit cytotoxic T cells. In some embodiments, the cysteine-modified PSMA polypeptides, including dimers thereof, can be used to generate an antibody that binds to native PSMA dimer and/or PSMA-expressed on a cancer cell with an avidity and/or binding affinity that is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 7-fold, 10-fold, 20-fold, 30-fold, 40-fold, 50-fold, 70-fold, 100-fold, 200-fold, 300-fold, 500-fold, 1000-fold or more greater than that exhibited by the antibody for PSMA in monomeric form.

The cysteine-modified PSMA polypeptides provided, and disulfide-bond-stabilized PSMA dimers thereof, comprise a cysteine-modified stalk region. As used herein, a “cysteine-modified stalk region” is a cysteine-modified version of the stalk region of the native PSMA protein (the stalk region of the native PSMA protein is the amino acid sequence set forth in SEQ ID NO: 5). The term “cysteine-modified” is intended to refer to any modification of the stalk region so that it contains one or more cysteine residues. Modifications of the stalk region, therefore, include the substitution of one or more of the residues of the stalk region with a cysteine and/or the insertion of one or more cysteine residues into the stalk region sequence.

One or more of the residues of the stalk region can be substituted with a cysteine. In an embodiment 1, 2 or 3 residues of the stalk region are substituted. Any of the eleven amino acids of the stalk region can be substituted. In one embodiment the residues of the stalk region that are substituted correspond to the residues at positions 1, 2, 3, 4, 5, 6 and/or 7 of the stalk region sequence. In another embodiment the substituted residues correspond to the residues at positions 1, 2, 3, 4 and/or 5. In still another embodiment the substituted residues correspond to the residues at positions 1, 2 and/or 3. In yet another embodiment one residue is substituted, and the substituted residue is the residue at position 1, 2, 3, 4 or 5 of the stalk region sequence. In another embodiment one residue is substituted, and the substituted residue is the residue at position 1, 2 or 3 of the stalk region sequence. In still another embodiment one residue is substituted, and the substituted residue is the residue at position 3.

One or more cysteine residues can be inserted into the stalk region sequence or at the amino or carboxy terminus of the stalk region. In one embodiment 1, 2 or 3 cysteine residues are inserted into the stalk region. The inserted cysteine residues can be inserted as a contiguous set of cysteines, or they can be inserted non-contiguously (i.e. at noncontiguous positions within the stalk region sequence or at the amino or carboxy terminus). For instance, when a set of cysteines is inserted “contiguously” into the stalk region, all of the cysteines are inserted between the same two residues (e.g., before the residue at position 1, after the residue at position 11, between the residues at positions 1 and 2, etc.). When the cysteines are inserted “non-contiguously”, each cysteine is separated by at least one residue of the stalk region from another cysteine. For example, one cysteine can be inserted between the residues at positions 1 and 2 of the stalk region and another cysteine can be inserted between the residues at positions 3 and 4. As another example, one cysteine can be inserted before the residue at position 1 and another cysteine can be inserted between the residues at positions 7 and 8 of the stalk region sequence. In some embodiments the cysteines are inserted between the residues at positions 1 and 2, 2 and 3, and/or 3 and 4 of the stalk region sequence.

The one or more cysteine residues that are inserted can be one or more cysteine residues alone, without any other amino acid residues, or they can be part of an amino acid insertion sequence that includes other amino acid residues. When the one or more cysteines are part of an amino acid insertion sequence, it is the amino acid sequence that is inserted. In one embodiment the amino acid insertion sequence is X¹ₙ-X²-X³-X⁴-X⁵-X⁶n, wherein X₁, X₂, X₃, X₄, X₅ and X₆ are each any amino acid, and wherein n is 0 or 1, provided that the amino acid insertion sequence contains at least one cysteine. In another embodiment the amino acid insertion sequence contains at least 2 cysteines. In still another embodiment the amino acid insertion sequence contains at least 3 cysteines. In yet another embodiment the amino acid insertion sequence contains at least 4 cysteines. In a further embodiment the amino acid insertion sequence contains 1, 2, 3 or 4 cysteines. In another embodiment the amino acid insertion sequence is a sequence of no more than 6 amino acids. In still another embodiment the amino acid insertion sequence is a sequence of 2, 3, 4, 5 or 6 amino acids. In another embodiment the amino acid insertion sequence is C-X¹ₙ-X²ₙ-C, wherein X¹ and X² are each any amino acid, n is 0, 1 or 2 and C is cysteine. In yet another embodiment X¹ and X² are each proline or serine. In a further embodiment X¹ and X² are each proline. In yet a further embodiment X¹ is proline and X² is serine. In still a further embodiment X¹ and X² are each cysteine. In some embodiments n is 1.

In yet another embodiment the insertion sequence comprises a cysteine residue and one, two, three, four or five other amino acids. In another embodiment the insertion sequence consists of a cysteine residue and one, two, three, four or five other amino acids. In still another embodiment the insertion sequence contains no more than six amino acid residues.

The inserted cysteine residues or sequences containing cysteine residues can be inserted anywhere within the stalk region sequence or at the amino or carboxy terminus of the stalk region sequence. In one embodiment the insertion occurs after the residue at position 1 but before the residue at position 11 of the stalk region. For instance, the insertions can occur between the residues at positions 1 and 2 of the stalk region. The insertions can also occur between the residues at positions 2 and 3, 3 and 4, 4 and 5, 5 and 6, 6 and 7, 7 and 8, 8 and 9, 9 and 10, and 10 and 11 of the stalk region. In another embodiment the insertion is before the amino acid at position 1. In yet another embodiment the insertion is after the residue at position 11. In still another embodiment the insertion is between the residues at positions 1 and 2, 2 and 3 or 3 and 4 of the stalk region sequence. In another embodiment the insertion is between the residues at positions 1 and 2 of the stalk region sequence.

The cysteine-modified stalk regions can in some embodiments include some combination of substitutions with and insertions of one or more cysteine residues as described above.

In some embodiments the cysteine-modified stalk region has a substitution at the residue corresponding to position 1 of the stalk region. The substitution can be a conservative substitution. The substitution of this residue, in some embodiments, is in addition to one or more cysteine substitutions and/or insertions as provided herein. The residue at this position can, for example, be modified with any amino acid that is not positively charged. Examples of amino acids that can substitute for the residue at position 1 of the stalk region sequence include glutamine, glutamic acid, aspartic acid, asparagine, cysteine, glycine or alanine.

In one embodiment, where the residue at position 1 of the stalk region is modified with a residue other than cysteine, one or more residues corresponding to the residues at positions 2-11 are substituted with a cysteine. In another embodiment, where the residue at position 1 of the stalk region is modified with a residue other than cysteine, one or more cysteines or a sequence containing one or more cysteines is inserted into the stalk region sequence or at the amino or carboxy terminues of the stalk region sequence.

The cysteine-modified PSMA polypeptides, and disulfide-bond-stabilized dimers thereof, can comprise a cysteine-modified stalk region and an amino acid sequence beginning with the amino acid residue at position 55 and ending with the amino acid residue at position 750 of SEQ ID NO: 1 (SEQ ID NO: 4) or a fragment thereof. Fragments of the amino acid sequence set forth as SEQ ID NO: 4 include fragments that begin at amino acid 1, 2, 3, 4, 5, 6, 7, 8, 9, 10, 11, 12, 13, 14, 15, 16, 17, 18, 19, 20, 25, 30, 35, 40, 45, 50, 75, 100, 150, 200, etc. of SEQ ID NO: 4 and end at amino acid 696 of SEQ ID NO: 4. Other fragments begin at amino acid 1 of SEQ ID NO: 4 and end at amino acid 695, 694, 693, 692, 691, 690, 689, 688, 687, 686, 685, 684, 683, 682, 681, 680, 677, 670, 650, 625, 600, 550, 500, etc. of SEQ ID NO: 4. Still other fragments include those that begin at amino acid 1, 2, 3, 4, 5, 6, 7, 8, 9, 10, 11, 12, 13, 14, 15, 16, 17, 18, 19, 20, 25, 30, 35, 40, 45, 50, 75, 100, 150, 200, etc. of SEQ ID NO: 4 and end at amino acid 695, 694, 693, 692, 691, 690, 689, 688, 687, 686, 685, 684, 683, 682, 681, 680, 677, 670, 650, 625, 600, 550, 500, etc. of SEQ ID NO: 4. The fragment of SEQ ID NO: 4 can have a size of at least about 25, 50, 100, 125, 150, 175, 200, 250, 300, 350, 400, 450, 500, 550, 600, 650 or 675 amino acids and every integer length therebetween. In some embodiments these fragments comprise amino acids 9-14, 78-83 or 428-433 of SEQ ID NO: 4 (these amino acids correspond to amino acids 63-68, 132-137 and 482-487 of the full-length PSMA sequence (SEQ ID NO: 1)). The cysteine-modified PSMA polypeptides can comprise any fragment of SEQ ID NO: 4 that with a cysteine-modified stalk region is capable of forming a PSMA polypeptide dimer as provided herein. Any portion of SEQ ID NO: 4 is included in this definition of a fragment of SEQ ID NO: 4.

The cysteine-modified PSMA polypeptides, which include dimers thereof, that comprise a cysteine-modified stalk region and the amino acid sequence of SEQ ID NO: 4, in one embodiment, generate antibodies that recognize native PSMA, PSMA-expressing cancer cells and/or elicit cytotoxic T cells that recognize PSMA-expressing cells. The cysteine-modified PSMA polypeptide can in one embodiment comprise a cysteine-modified stalk region and the amino acid sequence set forth as SEQ ID NO: 4. In another embodiment the PSMA polypeptide can comprise a cysteine-modified stalk region and amino acid residues 4-696 of the amino acid sequence set forth as SEQ ID NO: 4. In still another embodiment the PSMA polypeptide can comprise a cysteine-modified stalk region and amino acid residues 547-696 of the amino acid sequence set forth as SEQ ID NO: 4.

The cysteine-modified PSMA polypeptides provided, when in stabilized dimer form, can, in some embodiments, retain an activity of PSMA. The PSMA activity may be an enzymatic activity, such as folate hydrolase activity, NAALADase activity, dipeptidyl peptidase IV activity and γ-glutamyl hydrolase activity. Methods for testing the PSMA activity of PSMA polypeptide dimers are well known in the art (reviewed by O'Keefe et al. in: Prostate Cancer: Biology, Genetics, and the New Therapeutics, L. W. K. Chung, W. B. Isaacs and J. W. Simons (eds.) Humana Press, Totowa, N.J., 2000, pp. 307-326). In one embodiment the cysteine-modified PSMA polypeptides, when in stabilized dimer form, are recognized by an anti-PSMA antibody specific for native PSMA dimer. Examples of such antibodies as well as methods of assaying for antibody recognition of a particular antigen are provided in the Examples below and are known in the art.

Therefore, the cysteine-modified PSMA polypeptides provided can, in some embodiments, form homodimers, but they can also form heterodimers. As used herein, a “PSMA heterodimer” is a dimer of PSMA polypeptides that is composed of two different PSMA polypeptides. Examples include two PSMA polypeptides, where one is slightly longer than the other or where one has a conservative amino acid substitution and the other does not. The heterodimers provided herein, like homodimers, can be used to generate antibodies that bind, preferably specifically, to native PSMA dimer and/or PSMA-expressing cancer cells. In some embodiments the antibodies raised against the PSMA heterodimers recognize native PSMA dimer but not PSMA monomer. In still other embodiments these antibodies have greater specificity for native PSMA dimer rather than PSMA monomer. The heterodimers, like homodimers, can also be used to generate antibodies that elicit cytotoxic T cells.

The skilled artisan will realize that conservative amino acid substitutions may be made in the amino acid sequence of SEQ ID NO: 4 or the fragments described above to provide functional equivalents of SEQ ID NO: 4 or fragments thereof, i.e., modified versions that retain desired functional capabilities as compared to the non-modified version. These functional equivalents of SEQ ID NO: 4 or fragments thereof include those that when combined with a cysteine-modified stalk region are capable of associating to form disulfide-bond-stabilized dimers. Therefore, cysteine-modified PSMA polypeptides are also provided that comprise a cysteine-modified stalk region and a functional equivalent of SEQ ID NO: 4 or a fragment thereof. The functional equivalent of SEQ ID NO: 4 or a fragment thereof can be, in some embodiments, a conservatively substituted version of SEQ ID NO: 4 or a fragment thereof.

As used herein, a “conservative amino acid substitution” refers to an amino acid substitution which does not alter the relative charge or size characteristics of the protein in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Conservative amino-acid substitutions typically are made by alteration of a nucleic acid encoding a polypeptide. Conservatively substituted fragments include those with 1, 2, 3, 4, 5, 6, 7, 8, 9, 10, 15 or 20 substitutions. Such substitutions can be made by a variety of methods known to one of ordinary skill in the art. For example, amino acid substitutions may be made by PCR-directed mutation, site-directed mutagenesis, or by chemical synthesis of a gene encoding a polypeptide. Where amino acid substitutions are made to a small fragment, the substitutions can be made by directly synthesizing the peptide. The activity of a functional equivalent can be tested by cloning the gene encoding the altered polypeptide into a bacterial or mammalian expression vector, introducing the vector into an appropriate host cell, expressing the altered polypeptide, and testing for a functional capability. In general, functional equivalents include polypeptides which are modified specifically to alter a feature of the polypeptide unrelated to its physiological activity. For example, certain amino acids can be changed to enhance expression of a polypeptide by eliminating proteolysis by proteases in an expression system (e.g., dibasic amino acid residues in yeast expression systems in which KEX2 protease activity is present).

In certain embodiments, the functional equivalent of SEQ ID NO: 4 or a fragment thereof is encoded by a nucleic acid molecule that is highly homologous to the nucleic acid molecules that encode the non-modified version. Preferably the homologous nucleic acid molecule comprises a nucleotide sequence that is at least about 90% identical to a nucleotide sequence that encodes the non-modified polypeptide. More preferably, the nucleotide sequence is at least about 95% identical, at least about 97% identical, at least about 98% identical, or at least about 99% identical. The homology can be calculated using various, publicly available software tools well known to one of ordinary skill in the art. Exemplary tools include the BLAST system available from the website of the National Center for Biotechnology Information (NCBI) at the National Institutes of Health.

One method of identifying highly homologous nucleotide sequences is via nucleic acid hybridization. Thus the invention also includes functional equivalents encoded by nucleic acid molecules that hybridize under high stringency conditions to the nucleic acid molecules encoding a polypeptide of SEQ ID NO: 4 or fragments thereof. Identification of related sequences can also be achieved using polymerase chain reaction (PCR) and other amplification techniques suitable for cloning related nucleic acid sequences. Preferably, PCR primers are selected to amplify portions of a nucleic acid sequence of interest.

The term “high stringency conditions” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. One example of high-stringency conditions is hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68° C.

Functional equivalents of SEQ ID NO: 4 or fragments thereof are also intended to include homologous sequences from other species. For instance, PSMA has been found in other species, such as the pig (GenBank Accession Number 077564 (amino acid)) and rat (GenBank Accession Numbers U75973 (mRNA) and AAC53423 (amino acid)). Therefore, in one embodiment cysteine-modified polypeptides are provided that comprise a cysteine-modified stalk region and a fragment of PSMA from another species. In another embodiment the fragment of PSMA from another species is a fragment of the amino acid sequence of 077564 or AAC53423. In still another embodiment the fragment of PSMA from another species is the extracellular portion of the protein or some portion thereof.

Functional equivalents of SEQ ID NO: 4 or fragments thereof also include SEQ ID NO: 4 or fragments thereof with altered glycosylation. In one embodiment these functional equivalents can be produced by expressing SEQ ID NO: 4 or a fragment thereof in a cell that results in altered glycosylation. In one embodiment the cell is an insect cell. In another embodiment the cell is a bacterial cell. In still another-embodiment the cell is a mammalian cell. In one embodiment the cell is a non-human mammalian cell.

In some embodiments the functional equivalents provided when combined with a cysteine-modified stalk region are capable of forming disulfide-bond-stabilized dimers.

Methods of producing the functional equivalents of cysteine-modified PSMA polypeptides are also provided. In one embodiment the method comprises altering a nucleic acid encoding a cysteine-modified PSMA polypeptide as described herein and transfecting or transforming cells with a vector containing the altered nucleic acid. In one embodiment the nucleic acid is altered so that it codes for a conservative substitution of an amino acid. In another embodiment the nucleic acid is altered so that it codes for an insertion of one or more amino acid residues. In some embodiments the method further comprises harvesting and purifying the functionally equivalent cysteine-modified PSMA polypeptide expressed.

In another embodiment a method is provided which comprises transfecting or transforming cells with a vector encoding a cysteine-modified PSMA polypeptide, wherein the cells express the cysteine-modified PSMA polypeptide with altered glycosylation. In one embodiment the cells are insect cells. In some embodiments the method further comprises harvesting and purifying the cysteine-modified PSMA polypeptide with altered glycosylation that is expressed.

Functional equivalents, in some embodiments, retain a distinct functional capability of native PSMA. Functional capabilities which can be retained include the ability to form dimers, interaction with antibodies, interaction with other polypeptides or fragments thereof, and enzymatic activity. Therefore, functional equivalents can be selected according to certain properties. For example, one of ordinary skill in the art can prepare functional equivalents recombinantly and test them according to the desired functional capabilities.

Methods for altering polypeptide sequences are known to those of ordinary skill in the art and can be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Modifications are typically made to a nucleic acid which encodes a polypeptide. Mutations of a nucleic acid which encode a polypeptide preferably preserve the amino acid reading frame of the coding sequence, and preferably do not create regions in the nucleic acid which are likely to hybridize to form secondary structures, such a hairpins or loops, which can be deleterious to expression of the modified polypeptide.

Modifications can be made by selecting an amino acid substitution (e.g., one or more substitutions with a cysteine residue), or by random mutagenesis of a selected site in a nucleic acid which encodes the polypeptide. Modified polypeptides then can be expressed and tested for one or more activities (e.g., antibody binding, enzymatic activity, dimeric stability) to determine which mutation provides a modified polypeptide with the desired properties. Further mutations can be made to modified polypeptides (or to non-modified polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a polypeptide coding sequence or cDNA clone to enhance expression of the polypeptide. The foregoing procedures are well known to one of ordinary skill in the art. Further examples of the preparation of the cysteine-modified PSMA polypeptides described herein are provided below in the Examples.

Those of ordinary skill in the art will appreciate that the invention includes nucleic acids encoding the cysteine-modified PSMA polypeptides described herein. Also provided are compositions containing such nucleic acid molecules (e.g., nucleic acid vaccine compositions) as are methods of using the compositions (e.g., to stimulate an immune response, to produce cysteine-modified PSMA polypeptides, etc.).

As used herein, “codes for” or “encoding” refers to a region of a nucleotide sequence that encodes a polypeptide sequence. A coding region can include a region coding for a portion of a protein that is later cleaved off, such as a signal peptide.

The nucleic acid molecules that encode the cysteine-modified PSMA polypeptides provided can be DNA or RNA nucleic acids. The nucleic acid molecules can be comprised in a vector. The vector can be a plasmid (e.g., DNA plasmid) or viral vector. Numerous vector systems for expression of cysteine-modified PSMA polypeptides may be employed. For example, one class of vectors utilizes DNA elements which are derived from animal viruses such as bovine papilloma virus, polyoma virus, adenovirus, vaccinia virus, baculovirus, retroviruses (RSV, MMTV or MoMLV), Semliki Forest virus or SV40 virus.

Vaccine compositions, therefore, are provided comprising a cysteine-modified PSMA polypeptide, dimer thereof, or a nucleic acid delivery vehicle and a nucleic acid encoding a cysteine-modified PSMA polypeptide. The vaccine compositions can also include an adjuvant, cytokine and/or another therapeutic agent. Such compounds are described further below. In one embodiment the nucleic acid is capable of replicating in a cell of an animal or human being vaccinated. In one embodiment the replicated nucleic acid has as least a limited capacity to spread to other cells of the host and start a new cycle of replication. In another embodiment, the nucleic acid is non-replicating in an animal or human being being vaccinated. In one embodiment, the nucleic acid comprises a nucleic acid of a poxvirus, a herpes virus and/or an adenovirus. In another embodiment, the nucleic acid comprises the nucleic acid of an alphavirus including but not limited to Venezuelan equine encephalitis (VEE) virus, Semliki Forest Virus, Sindbis virus, and the like. In still another embodiment, the nucleic acid delivery vehicle is a virus particle, such as a VEE virus particle, Semliki Forest Virus particle, a Sindbis virus particle, a pox virus particle, a herpes virus particle or an adenovirus particle. The vectors used are designed, in some embodiments, to express the cysteine-modified PSMA polypeptides in eukaryotic cells as well as efficiently secrete the polypeptides.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagenids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Examples of vectors that may be used include but are not limited to pcDNA3.1 (Invitrogen; Cat. #V790-20), pCI mammalian expression vector (Promega, Madison, Wis.; Cat. #E1731) and pCMV-script (Stratagene, La Jolla, Calif.; Cat. # 212220). Cells are genetically engineered by the introduction into the cells of heterologous DNA or RNA. The heterologous DNA or RNA is placed under operable control of transcriptional elements to permit the expression of the heterologous DNA in the host cell.

The vectors can be used to transform or transfect host cells for producing polypeptides. In some embodiments the vector is operably linked to a promoter. Therefore, host cells transformed or transfected with the vectors are provided as are methods of producing polypeptides by transforming or transfecting cells with these vectors. The polypeptides encoded by the nucleic acid molecules described and compositions that include these polypeptides are also provided.

Once the expression vector or DNA sequence containing the constructs has been prepared for expression, the expression vectors can be transfected or introduced into an appropriate cell host, e.g., mammalian cell host. Various techniques may be employed to achieve this, such as, for example, protoplast fusion, calcium phosphate precipitation, electroporation, retroviral transduction, or other conventional techniques. Methods and conditions for culturing the resulting cells and for recovering the cysteine-modified PSMA polypeptides so produced are well known to those skilled in the art, and may be varied or optimized depending upon the specific expression vector and mammalian host cell employed.

In accordance with the claimed invention, the host cells for expressing the cysteine-modified PSMA polypeptides of this invention include mammalian cell lines. Mammalian cell lines include, for example, monkey kidney CV1 line transformed by SV40 (COS-7); human embryonic kidney line 293; baby hamster kidney cells (BHK); Chinese hamster ovary-cells-DHFR⁺ (CHO); Chinese hamster ovary-cells DHFR⁻ (DXB11); monkey kidney cells (CV1); African green monkey kidney cells (VERO-76); human cervical carcinoma cells (HELA); canine kidney cells (MDCK); human lung cells (W138); human liver cells (Hep G2); mouse mammary tumor (MMT 060562); mouse cell line (C127); and myeloma cell lines.

Other eukaryotic expression systems utilizing non-mammalian vector/cell line combinations can be used to produce the cysteine-modified PSMA polypeptides. These include, but are not limited to, baculovirus vector/insect cell expression systems and yeast shuttle vector/yeast cell expression systems.

In another embodiment, the present invention provides host cells, both prokaryotic and eukaryotic, transformed or transfected with, and therefore including, the vectors provided. The host cells include those described above transformed or transfected with the described vectors.

The nucleic acids and polypeptides provided in some embodiments are isolated. As used herein, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated polypeptides may be, but need not be, substantially pure. The term “substantially pure” means that the polypeptides are essentially free of other substances with which they may be found in nature or in vivo systems to an extent practical and appropriate for their intended use. Substantially pure polypeptides may be produced by techniques well known in the art. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, i.e. isolated from other polypeptides.

Preferred systems for expression are provided in the Examples and will also be known to those of ordinary skill in the art. The subsequent purification of the peptides may be accomplished by any of a variety of standard means known in the art. Purification practices known to those of ordinary skill in the art can, therefore, be used to prepare compositions of cysteine-modified PSMA polypeptides wherein at least 25%, 50%, 75%, 80%, 85%, 90%, 95% or more of the cysteine-modified PSMA polypeptides are in dimer form. In one embodiment at least 75% of the cysteine-modified PSMA polypeptides are in dimer form. In another embodiment at least 90% of the cysteine-modified PSMA polypeptides are in dimer form.

Since the PSMA polypeptides provide herein can contain a cysteine-modified stalk region and a fragment of SEQ ID NO: 4, the PSMA polypeptides in some embodiments are fusion polypeptides. To make a fusion polypeptide in accordance with the invention, a nucleic acid molecule is generated that encodes a fragment of SEQ ID NO: 4 and a cysteine-modified stalk region. Such fusion proteins contain a fragment of SEQ ID NO: 4 and a cysteine-modified stalk region, operatively attached. The fusion proteins may, in some embodiments, also include additional peptide sequences, such as peptide spacers which operatively attach the fragment of SEQ ID NO: 4 and cysteine-modified stalk region, as long as such additional sequences do not appreciably affect a desired function of the fusion polypeptide (e.g., the ability to form dimers.) In other embodiments no additional peptide sequences are included. Other fusion arrangements will be known to one of ordinary skill in the art.

To express the fusion protein, the nucleic acid encoding the fusion protein is inserted into an expression vector in accordance with standard methods, for stable expression of the fusion protein. The fusion protein can be isolated and purified from the cells or culture supernatant using standard methodology, such as a PSMA affinity column.

Methods of producing cysteine-modified PSMA polypeptides are, therefore, also provided in one aspect of the invention. Such methods in one embodiment include the steps of modifying a nucleic acid molecule that encodes a PSMA polypeptide comprising the stalk region of PSMA so that the nucleic acid molecule codes for a cysteine residue within the stalk region sequence and transforming or transfecting cells with a vector containing the modified nucleic acid molecule. The nucleic acid molecule can be modified so that its sequence codes for a cysteine substitution within the stalk region sequence. The nucleic acid molecules can also be modified so that its sequence codes for a cysteine insertion within the stalk region sequence. In one embodiment the codon that codes for a cysteine is tgt but is not necessarily so. It will be recognized by those of ordinary skill in the art that due to the degeneracy of the genetic code other codons that code for a cysteine can be used. Also provided in one aspect of the invention is a polypeptide produced by the foregoing method as is the modified nucleic acid molecule used in the foregoing method.

It has further been discovered that the presence of an anti-apoptotic agent, such as dextran sulfate, in the expression media resulted in the higher expression of PSMA polypeptides. Additionally, the presence of polyethylene glycol (PEG) in the expression media resulted in a raised dimer to monomer ratio. Therefore, compositions comprising transformed or transfected cells, preferably cells transformed or transfected with polypeptide-encoding vectors, and dextran sulfate and/or PEG are also provided. Methods of producing polypeptides, such as PSMA polypeptides, with expression media containing an anti-apoptotic agent and/or PEG are likewise provided. Such methods include transforming or transfecting cells with a vector encoding a polypeptide and contacting the cells with media comprising an anti-apoptotic agent and/or PEG. The PEG may be of a molecular weight of 2000, 3000, 4000, 6000 or 8000. In one embodiment the PEG is PEG 8000. Anti-apoptotic agents that enhance the expression of polypeptides include, but are not limited to, dextran sulfate, tropolone, caspase inhibitors and the BCL2 gene product.

The compositions provided can be used to stimulate an immune response (i.e., elicit or enhance an immune response) to the cysteine-modified PSMA polypeptides, native PSMA dimer and/or cells expressing PSMA, such as PSMA-expressing cancer cells. Therefore, methods are also provided for stimulating an immune response, whereby a composition comprising a cysteine-modified PSMA polypeptide, or dimer thereof, or a nucleic acid that encodes a cysteine-modified PSMA polypeptide, as provided herein, is administered to a subject in an amount effective to stimulate an immune response. In one embodiment the immune response includes both a B cell and cytotoxic T cell response. Such methods can further include the administration of one or more other doses of a composition comprising full-length PSMA polypeptide or a fragment thereof, rsPSMA in monomeric or dimeric form, the native protein in dimeric form or a nucleic acid that encodes one of these polypeptides. In another embodiment the methods further include the administration of one or more other doses of a composition comprising a cysteine-modified PSMA polypeptide in monomeric or dimeric form or a nucleic acid that encodes a cysteine-modified PSMA polypeptide. In all of the embodiments of these methods at least one dose of a composition comprising a cysteine-modified PSMA polypeptide in monomeric or dimeric form or a nucleic acid molecule that encodes a cysteine-modified PSMA polypeptide is administered to the subject. The composition comprising a cysteine-modified PSMA polypeptide or a nucleic acid molecule that encodes it can be administered as an initial or a subsequent dose or concomitantly with a dose of another polypeptide or nucleic acid composition as described above.

In these methods multiple doses can be administered to a subject concomitantly or they are administered at different times. Generally, there will be an initial dose followed by a booster dose. In one embodiment the initial dose will be of a composition comprising a nucleic acid as described above. In another embodiment the booster dose will be a composition comprising a polypeptide as described above. In one embodiment the polypeptide is full-length PSMA polypeptide or a fragment thereof, rsPSMA in monomeric or dimeric form, the native protein in dimeric form or a cysteine-modified PSMA polypeptide in monomeric or dimeric form. In another embodiment the polypeptide is a cysteine-modified PSMA polypeptide. In another embodiment the polypeptide is native dimeric PSMA or rsPSMA in dimer form. In still another embodiment the initial dose composition comprises one or more cells that express a polypeptide as described above, such as, for example, native PSMA dimer, rsPSMA dimer or a cysteine-modified PSMA polypeptide.

The potential exists to tailor the nature of the immune responses by priming (with an initial dose) and then delivering subsequent boosts with the same or different forms of the antigen or by delivering the antigen to different immunological sites and/or antigen presenting cell populations. Indeed, the ability to induce preferred type-1 or type-2 like T-helper responses or to additionally generate specific responses at mucosal and/or systemic sites can be foreseen with such an approach. Prime-boost protocols are described in U.S. Pat. No. 6,210,663 B1 and WO 00/44410. Such protocols are expressly incorporated herein by reference.

In one embodiment, the priming (i.e., initial) composition (or dose) is preferably, in some embodiments, administered systemically. This systemic administration includes any parenteral routes of administration characterized by physical breaching of a tissue of a subject and administration of the pharmaceutical composition through the breach in the tissue. In particular, parenteral administration is contemplated to include, but is not limited to, intradermal, transdermal, subcutaneous, intraperitoneal, intravenous, intraarterial, intramuscular, or intrasternal injection, intravenous, interaarterial, or kidney dialytic infusion techniques, and so-called “needleless” injections through tissue. Preferably, in some embodiments, the systemic, parenteral administration is intramauscular injection. In another embodiment, the priming composition is administered at a site of administration including the intranasal, oral, vaginal, intratracheal, intestinal or rectal mucosal surfaces.

The priming composition may be administered at various sites in the body in a dose-dependent manner. The invention is not limited to the amount or sites of injection(s) or to the pharmaceutical carrier, nor to this immunization protocol. Rather, the priming step encompasses treatment regimens which include a single dose or dosage which is administered hourly, daily, weekly, or monthly, or yearly.

Preferably, but not limited to, a boosting composition is administered about 2 to 27 weeks after administering the priming composition to a mammalian subject. The administration of the boosting composition is accomplished using an effective amount of a boosting composition containing or capable of delivering the same antigen (in the same or different form) as administered by the priming composition.

In another example, one embodiment of a priming and/or boosting composition is a replication competent or replication defective recombinant virus containing a DNA sequence encoding a polypeptide as described above, such as full-length PSMA, rsPSMA or a cysteine-modified PSMA polypeptide. In another embodiment, the priming and/or boosting composition is a nonreplicating alphavirus comprising a nucleic acid molecule encoding a polypeptide described herein or a nonreplicating vaccine replicon particle derived from an alphavirus. Adenoviruses, which naturally invade their host through the airways, infect cells of the airways readily upon intranasal application and induce a strong immune response without the need for adjuvants. In another embodiment the priming and/or boosting composition comprises a replication defective recombinant adenovirus.

Another example of a priming and/or boosting composition is a bacterial recombinant vector containing a DNA sequence encoding the antigen in operable association with regulatory sequences directing expression of the antigen in tissues of the mammal. One example is a recombinant BCG vector. Other examples include recombinant bacterial vectors based on Salmonella, Shigella, and Listeria, among others.

Still another example of a priming and/or boosting composition is a naked DNA sequence encoding the antigen in operable association with regulatory sequences directing expression of the antigen in tissues of the mammal but containing no additional vector sequences.

In still additional embodiments, the priming and/or boosting composition can include a composition which comprises a polypeptide as described above or cells transformed or transfected with a nucleic acid molecule encoding such a polypeptide.

All of the priming and boosting compositions can, in some embodiments, include adjuvants and/or cytokines. The priming and boosting compositions can in other embodiments include additional therapeutic agents. Further, the priming and boosting compositions can contain pharmaceutically suitable or physiologically acceptable carriers.

Also provided herein is a vaccine which comprises a prophylactically effective amount of an isolated nucleic acid encoding a cysteine-modified PSMA polypeptide. The invention also provides a vaccine which comprises a prophylactically effective amount of a cysteine-modified PSMA polypeptide encoded by the isolated nucleic acid. A prophylactically effective amount of the vaccine may be determined according to methods well known to those skilled in the art. As used herein “prophylactically effective amount” refers to a dose and dosing schedule sufficient to reduce the likelihood of a subject to develop cancer, such as prostate cancer, or to lessen the severity of the disease in subjects who do develop cancer.

In these methods any mode of administration known to those of ordinary skill in the art can be utilized. For example, the initial/priming and booster doses of the compositions provided can be administered by intravenous, intramuscular, subcutaneous, parenteral, spinal, intradermal or epidermal administration. The initial and booster doses can be administered with the same or different mode of administration.

The initial, and optional booster doses, can be administered to a subject that is at risk of, has or has been treated for cancer. Such cancers are intended to include any cancer in which PSMA expression is associated therewith. Such cancers include, therefore, prostate cancer as well as other cancers as described herein. The initial, and optional booster doses, can also be administered to a subject from which antibodies can be harvested. Therefore, methods are provided, which further include the step of harvesting antibodies produced as a result of the stimulated immune response.

The compositions provided herein can be used to treat a subject that has or is at risk of having a cancer. Methods of treating cancer in a subject are likewise provided. Such methods include the administration of a therapeutically effective amount of a composition provided herein effective in treating a cancer. The cancers include prostate, breast, bladder, urothelial, pancreatic, lung, liver, colon, rectal and kidney cancer; melanomas and sarcomas. The cancers also include cancers of the female reproductive tract, such as ovarian, cervical, endometrial, uterine, vaginal, vulvar or pelvic cancers and gestational trophoblastic tumors. The cancers further include childhood cancers, such as leukemias, neuroblastomas, brain cancers, lymphomas, Wilm's tumors, bone cancers, retinoblastomas, rhabdomyosarcomas, and ovarian germ cell tumors. The cancer cells can be cells of a primary tumor or can be those of a metastatic tumor. For example, the subject can be one with cancerous tissue of metastatic bone marrow or cancerous tissue of metastatic lymph nodes. The subjects that can be treated with the compositions and methods provided can be any subject in which there are cancer cells or neovasculature cells of a cancer/tumor that express PSMA.

The compositions provided herein can be administered to a subject who has received conventional cancer therapy or in combination with a conventional cancer therapy. Current standard or conventional treatments for cancer, such as prostate cancer, include surgery, radiation, cryosurgery, thermotherapy, hormone treatment and chemotherapy. Subjects receiving one or more of the standard treatments may be referred to as treatment-experienced subjects. Hormone therapy includes treatment with one or more of the following modalities: a leutinizing hormone-releasing hormone agonist such as leuprolide, goserelin or buserelin; an antiandrogen, such as flutaminde or bicalutamide; a drug that prevents adrenal glands from making androgens, such as ketoconazole or aminoglutethimide; estrogens; and orchiectomy (castration). Chemotherapy may use any chemotherapeutic/antineoplastic agent known in the art. In some embodiments the chemotherapeutic agent is a taxane, such as paclitaxel (Taxol®) or docetaxel (Taxotere®). Other chemotherapeutic agents include DNA damaging agents and these include topoisomerase inhibitors (e.g., etoposide, ramptothecin, topotecan, teniposide, mitoxantrone), anti-microtubule agents (e.g., vincristine, vinblastine), anti-metabolite agents (e.g., cytarabine, methotrexate, hydroxyurea, 5-fluorouracil, floxuridine, 6-thioguanine, 6-mercaptopurine, fludarabine, pentostatin, chlorodeoxyadenosine), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C). Chemotherapeutic agents also include annonaceous acetogenins; asimicin; rolliniastatin; guanacone, squamocin, bullatacin; squamotacin; taxanes such as paclitaxel and docetaxel; gemcitabine; methotrexate FR-900482; FK-973; FR-66979; FK-317; 5-FU; FUDR; FdUMP; discodermolide; epothilones; vinorelbine; meta-pac; irinotecan; SN-38; 10-OH campto; flavopiridol; mithramycin; capecitabine; cytarabine; 2-Cl-2′deoxyadenosine; Fludarabine-PO₄; mitozolomide; Pentostatin; Tomudex; pemetrexed; erlotinib; adriamycin; aldesleukin, asparaginase, bleomycin; bleomycin sulfate, carboplatin, chlorambucil, cisplatin, cladribine, cyclophosphamide, cytarabine, dacarbazine, dactinomycin, daunorubicin hydrochloride, docetaxel, doxorubicin, doxorubicin hydrochloride, epirubicin hydrochloride, etoposide, etoposide phosphate, floxuridine, fludarabine, fluorouracil, gemcitabine, gemcitabine hydrochloride, hydroxyurea, idarubicin hydrochloride, ifosfamide, interferons, interferon-α2a, interferon-α2b, interferon-αn3, interferon-α1b, interleukins, irinotecan, mechlorethamine hydrochloride, melphalan, mercatopurine, methotrexate, methotrexate sodium, mitomycin, mitomycin C; mitoxantrone, paclitaxel, pegaspargase, pentostatin, prednisone, profimer sodium, procabazine hydrochloride, taxol, taxotere, tenipo side, topotecan; topotecan hydrochloride, vinblastine; vinblastine sulfate, vincristine; vincristine sulfate and vinorelbine tartrate.

Chemotherapy may be used in combination with an anti-inflammatory compound such as a corticosteroid. Corticosteroids include cortisone, hydrocortisone, prednisone, prednisolone, triamcinolone, methylprednisolone, dexamethasone, betamethasone and the like. A preferred anti-inflammatory compound, in some embodiments, is prednisone.

Other therapeutic modalities that may be used in combination with the compositions provided include the use of other vaccines and immunotherapies. In one embodiment subjects amenable to treatment using the compositions provided include those who have not received conventional cancer treatment. In another embodiment subjects amenable to treatment using the compositions provided include those who have evidence of cancer despite having received one or more conventional cancer therapies. Subjects therefore can include patients with biochemically progressive prostate cancer such as non-castrate patients (serum testosterone greater than or equal to 180 ng/mL). In some embodiments these patients have received definitive primary therapy such as prostatectomy or radiation. Subjects can also include castrate patients (serum testosterone less than 50 ng/mL), who in some embodiments have completed a course of hormonal therapy. Subjects can also include patients having radiographic evidence of disease progression. In one embodiment such a treatment regimen is indicated in hormone-refractory prostate cancer patients. The subject can also be a non-castrate patient who has, in some embodiments, received primary therapy, such as prostatectomy and/or radiation therapy.

Compositions of the invention, therefore, can be administered in combination therapy, i.e., combined with other therapeutic agents, such as those described herein. For example, the combination therapy can include a composition of the present invention with at least one anti-tumor agent, chemotherapeutic agent, immunomodulator, immunostimulatory agent, or other conventional therapy. The therapeutic agent can, in some embodiments, be bound or conjugated to an anti-PSMA antibody.

Therapeutic agents include antitumor agents, such as cytotoxic agents and agents that act on tumor neovasculature. Cytotoxic agents include cytotoxic radionuclides, chemical toxins, chemotherapeutic agents and protein toxins. Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents include dolastatins (U.S. Pat. Nos. 6,034,065 and 6,239,104) and derivatives thereof. Other agents include dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, G. R. et al., Anticancer Drug Des. 13(4):243-277, 1998; Woyke, T. et al., Antimicrob. Agents Chemother. 45(12):3580-3584, 2001), and aurastatin E and the like. Toxins also include poisonous lectins, plant toxins such as ricin, abrin, modeccin, botulina and diphtheria toxins.

Agents that act on the tumor vasculature include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). Antiangiogenic agents also include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD1839), Marimastat, Metastat (Col-3), Neovastat, Octreotide, Paclitaxel, Penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), Tetrathiomolybdate, Thalidomide, TNP-470 and Vitaxin. Additional antiangiogenic agents are described by Kerbel, J. Clin. Oncol. 19(18s):45s-51s, 2001, which is incorporated by reference herein.

In some embodiments the various compositions/therapeutics can be administered concomitantly. In other embodiments the compositions/therapeutics are administered separately (prior to or subsequent to each other). For instance, a composition can be administered to such a subject at some time subsequent to a conventional cancer therapy. Conventional cancer therapy, such as for prostate cancer, includes one or more of the following: surgery, radiation, cryosurgery, thermotherapy, hormone treatment, chemotherapy, etc. In one embodiment the therapy received prior to administration of a composition as provided herein is at least prostatectomy and/or radiation. In another embodiment the therapy received prior to administration of a composition as provided herein is at least castration and hormonal therapy. In yet another embodiment the therapy received prior to administration is at least chemotherapy. In one embodiment for prostate cancer the chemotherapy is the administration of the chemotherapeutic agent, docetaxel, alone or in combination with an anti-inflammatory compound. The anti-inflammatory compound in one embodiment is prednisone.

Therefore, in some embodiments compositions and methods are provided for treating patients with a composition provided that is administered concomitantly with, subsequent to, or prior to conventional cancer therapy. In one such embodiment the methods provided include the administration of docetaxel (75 mg/m² q3 weeks) plus the anti-inflammatory agent, prednisone (5 mg po bid), concomitantly with, subsequent to, or prior to the administration of a composition as provided herein.

Treatment in accordance with the present invention can be effectively monitored with clinical parameters such as serum prostate specific antigen and/or pathological features of a patient's cancer, including stage, Gleason score, extracapsular, seminal, vesicle or perineural invasion, positive margins, involved lymph nodes, etc. Alternatively, these parameters can be used to indicate when such treatment should be employed.

The compositions and methods provided can include adjuvants/adjuvant administration. Adjuvants are well known in the art. An adjuvant is a substance which potentiates the immune response. Specific examples of adjuvants include monophosphoryl lipid A (MPL, SmithKline Beecham); saponins, including QS-7, QS-17, QS-18, QS-21 (Antigenics, New York, N.Y.; U.S. Pat. Nos. 6,524,584 and 6,645,495); saponin-based adjuvants, such as SaponImmune™ (GPI-0100) Series (Galenica Pharmaceuticals, Birmingham, Ala.; U.S. Pat. Nos. 5,977,081 and 6,080,725) and chemically modified saponins (Galenica Pharmaceuticals, U.S. Pat. No. 6,262,029); polysaccharide-based adjuvants, such as PolysaccImmune™ (GPI-0200) Series (Galenica Pharmaceuticals); synthetic adjuvants, such as SynthImmune™ (GPI-0300) Series (Galenica Pharmaceuticals); biodegradable particles composed of poly-lactide-co-glycolide (PLG) or other similar polymers; immunostimulatory oligonucleotides (e.g., CpG oligonucleotides described by Kreig et al., Nature 374:546-9, 1995); incomplete Freund's adjuvant; complete Freund's adjuvant; vitamin E and various water-in-oil emulsions prepared from biodegradable oils such as squalene and/or tocopherol; montanide, such as MONTANIDE ISA51 and MONTANIDE ISA720, which are water-in-oil emulsions provided by Seppic (Paris, France); Quil A; micellular mixtures of Quil A and cholesterol known as immunostimulating complexes (ISCOMS); MPL and cell wall skeleton from mycobacterium combinations such as ENHANZYN™ (Corixa, Seattle, Wash.); RC-529 (Corixa); RC-552 (Corixa); CRL-1005; L-121; alpha-galactosylceramide (Fujii et al., J. Exp. Med., 2003, July 21; 198(2): 267-79); aluminum or iron oxide beads and combinations thereof. Other specific examples of adjuvants include QS-21 fractions, such as crude QA-21; a QA-21H form; QA-21-V1; QA-21-V2; a combination of QA-21-V1 and QA-21-V2; and chemically modified forms or combinations thereof. Preferred adjuvants, in some embodiments, include alum and QS-21.

Other agents which can assist in the stimulation of an immune response in a subject can also be included in the compositions and methods provided. For example, cytokines are also useful in vaccination protocols as a result of their lymphocyte regulatory properties. Many cytokines useful for such purposes will be known to one of ordinary skill in the art, including interleukin-2 (IL-2); IL-4; IL-5; IL-12, which has been shown to enhance the protective effects of vaccines (see, e.g., Science 268: 1432-1434, 1995); GM-CSF; IL-15; IL-18; combinations thereof, and the like. Chemokines are useful in increasing immune responses and include, but are not limited to, SLC, ELC, MIP3α, MIP3β, IP-10, MIG and combinations thereof. The compositions and methods provided, therefore, can include combinations of adjuvants, cytokines and/or chemokines/adjuvant, cytokine and/or chemokine administration.

The compositions provided can also be used to immunize an animal for the purpose of raising antibodies to the cysteine-modified PSMA polypeptides provided, native PSMA dimer and/or PSMA expressed on cells, such as cancer cells. Methods of generating antibodies are, therefore, also provided.

As used herein, the term “antibody” refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody as used herein, refers to one or more portions of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). Furthermore, although the two domains of the Fv fragment, V and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

The antibodies that can be generated with the compositions provided can be polyclonal, monoclonal, or a mixture of polyclonal and monoclonal antibodies. The antibodies can be produced by a variety of techniques well known in the art. Procedures for raising polyclonal antibodies are well known. For example, polyclonal antibodies are raised by administering a composition provided subcutaneously to New Zealand white rabbits which have first been bled to obtain pre-immune serum. The composition can be injected at a total volume of 100 μl per site at six different sites, typically with one or more adjustments. The rabbits are then bled two weeks after the first injection and periodically boosted three times every six weeks. A sample of serum is collected 10 days after each boost. Polyclonal antibodies are recovered from the serum, preferably by affinity chromatography to capture the antibody. This and other procedures for raising polyclonal antibodies are disclosed in E. Harlow, et. al., editors, Antibodies: A Laboratory Manual (1998), which is hereby incorporated by reference.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired antigen. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. Mouse lymphocytes give a higher percentage of stable fusions with the mouse myeloma lines described herein. Of these, the BALB/c mouse is preferred. However, other mouse strains, rabbit, hamster, sheep and frog may also be used as hosts for preparing antibody-producing cells. See; Goding (in Monoclonal Antibodies: Principles and Practice, 2d ed., pp. 60-61, Orlando, Fla., Academic Press, 1986). In particular, mouse strains that have human immunoglobulin genes inserted in the genome (and which cannot produce mouse immunoglobulins) are preferred. Examples include the HuMAb mouse strains produced by Medarex/GenPharm International, and the XenoMouse strains produced by Abgenix. Such mice produce fully human immunoglobulin molecules in response to immunization.

Those antibody-producing cells that are in the dividing plasmablast stage fuse preferentially. Somatic cells may be obtained from the lymph nodes, spleens and peripheral blood of antigen-primed animals, and the lymphatic cells of choice depend to a large extent on their empirical usefulness in the particular fusion system. The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Alternatively, human somatic cells capable of producing antibody, specifically B lymphocytes, are suitable for fusion with myeloma cell lines. While B lymphocytes from biopsied spleens, tonsils or lymph nodes of an individual may be used, the more easily accessible peripheral blood B lymphocytes are preferred. In addition, human B cells may be directly immortalized by the Epstein-Barr virus (Cole et al., 1995, Monoclonal Antibodies and Cancer Therapy, Alan R. Liss, Inc., pp. 77-96). Although somatic cell hybridization procedures are preferred, in principle, other techniques for producing monoclonal antibodies can be employed such as viral or oncogenic transformation of B lymphocytes.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference).

The compositions provided can be used to generate antibodies or antigen-binding fragments thereof selected for their ability to bind cells expressing PSMA. In order to demonstrate binding of monoclonal antibodies to cells expressing PSMA, flow cytometry can be used. For example, cell lines expressing PSMA (grown under standard growth conditions) or prostate cancer cells that express PSMA are mixed with various concentrations of monoclonal antibodies in PBS containing 0.1% Tween 80 and 20% mouse serum, and incubated at 37° C. for 1 hour. After washing, the cells are reacted with fluorescein-labeled anti-human IgG secondary antibody (if human anti-PSMA antibodies were used) under the same conditions as the primary antibody staining. The samples can be analyzed by a fluorescence activated cell sorter (FACS) instrument using light and side scatter properties to gate on single cells. An alternative assay using fluorescence microscopy may be used, in addition to or instead of, the flow cytometry assay. Cells can be stained exactly as described above and examined by fluorescence microscopy. This method allows visualization of individual cells, but may have diminished sensitivity depending on the density of the antigen.

Binding of the antibody or antigen-binding fragment thereof to cells expressing PSMA can inhibit the growth of the cells or mediate cytolysis of the cells; therefore, the compositions provided can be used to generate such antibodies. Cytolysis can be complement mediated or can be mediated by effector cells. In one embodiment, the cytolysis is carried out in a living organism, preferably a mammal, and the live cell is a cancer/tumor cell.

The testing of antibody cytolytic activity in vitro by chromium release assay can provide an initial screening prior to testing in vivo models. This testing can be carried out using standard chromium release assays. Briefly, polymorphonuclear cells (PMN), or other effector cells, from healthy donors can be purified by Ficoll Hypaque density centrifugation, followed by lysis of contaminating erythrocytes. Washed PMNs can be suspended in RPMI supplemented with 10% heat-inactivated fetal calf serum and mixed with ⁵¹Cr labeled cells expressing PSMA, at various ratios of effector cells to tumor cells (effector cells:tumor cells). Purified anti-PSMA IgGs can then be added at various concentrations. Irrelevant IgG can be used as negative control. Assays can be carried out for 0-120 minutes at 37° C. Samples can be assayed for cytolysis by measuring ⁵¹Cr release into the culture supernatant. Anti-PSMA monoclonal antibodies can also be tested in combinations with each other to determine whether cytolysis is enhanced with multiple monoclonal antibodies. Antibodies which bind to PSMA also can be tested in an in vivo model (e.g., in mice) to determine their efficacy in mediating cytolysis and killing of cells expressing PSMA, e.g., cancer/tumor cells.

The compositions provided can, in some embodiments, be used to generate antibodies or antigen-binding fragments thereof that bind to a conformational epitope within the extracellular domain of PSMA. To determine if selected anti-PSMA antibodies bind to conformational epitopes, each antibody can be tested in assays using native protein (e.g., non-denaturing immunoprecipitation, flow cytometric analysis of cell surface binding) and denatured protein (e.g., Western blot, immunoprecipitation of denatured proteins). A comparison of the results will indicate whether the antibodies bind conformational epitopes. Antibodies that bind to native protein but not denatured protein are those antibodies that bind conformational epitopes, and are preferred antibodies, in some embodiments.

In another embodiment, the compositions can be used to generate antibodies or antigen-binding fragments thereof that bind to a dimer-specific epitope on PSMA. Generally, antibodies or antigen-binding fragments thereof which bind to a dimer-specific epitope preferentially bind the PSMA dimer rather than the PSMA monomer. To determine if the selected human anti-PSMA antibodies bind preferentially (i.e., selectively and/or specifically) to a PSMA dimer, each antibody can be tested in assays (e.g., immunoprecipitation followed by Western blotting) using native dimeric PSMA protein and dissociated monomeric PSMA protein. A comparison of the results will indicate whether the antibodies bind preferentially to the dimer or to the monomer. Antibodies that bind to the PSMA dimer but not to the monomeric PSMA protein, in some embodiments, are preferred antibodies.

The cysteine-modified PSMA polypeptides as described herein have a number of other uses. The cysteine-modified PSMA polypeptides are useful for testing compounds that modulate PSMA enzymatic activity or PSMA dimerization. The cysteine-modified PSMA polypeptides, including dimers thereof, can be used to isolate antibodies that selectively bind PSMA, including those selective for conformational epitopes, those selective for binding native PSMA dimer and those that selectively modulate an enzymatic activity of PSMA.

Compounds that selectively modulate an enzymatic activity of PSMA include agents that inhibit or enhance at least one enzymatic activity of PSMA, such as NAALADase activity, folate hydrolase activity, dipeptidyl dipeptidase IV activity, γ-glutamyl hydrolase activity or combinations thereof.

Thus methods of screening for agents are provided in accordance with the invention. The methods can include mixing a candidate agent with an cysteine-modified PSMA polypeptide dimer to form a reaction mixture, thereby contacting the cysteine-modified PSMA polypeptide dimer with the candidate agent. The methods also include adding a substrate for the cysteine-modified PSMA polypeptide dimer to the reaction mixture, and determining the amount of a product formed from the substrate by the cysteine-modified PSMA polypeptide dimer. Such methods are adaptable to automated, high-throughput screening of compounds. A decrease in the amount of product formed in comparison to a control is indicative of an agent capable of inhibiting at least one enzymatic activity of PSMA. An increase in the amount of product formed in comparison to a control is indicative of an agent capable of enhancing at least one enzymatic activity of PSMA.

The reaction mixture comprises a candidate agent. The candidate agent is preferably an antibody, a small organic compound, or a peptide, and accordingly can be selected from combinatorial antibody libraries, combinatorial protein libraries or small organic molecule libraries. Typically, a plurality of reaction mixtures are run in parallel with different agent concentrations to obtain a different response to the various concentrations. Typically, one of these concentrations serves as a negative control, i.e., at zero concentration of agent or at a concentration of agent below the limits of assay detection.

Candidate agents encompass numerous chemical classes, although typically they are organic compounds, proteins or antibodies (and fragments thereof that bind antigen). In some embodiments, the candidate agents are small organic compounds, i.e., those having a molecular weight of more than 50 yet less than about 2500, preferably less than about 1000 and, more preferably, less than about 500. Candidate agents comprise functional chemical groups necessary for structural interactions with polypeptides and/or nucleic acids, and typically include at least an amine, carbonyl, hydroxyl, or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random or non-random peptides, combinatorial libraries of proteins or antibodies, and the like. Alternatively, libraries of natural compounds in the form of bacterial, fungal, plant, and animal extracts are available or readily produced. Additionally, natural and synthetically produced libraries and compounds can readily be modified through conventional chemical, physical, and biochemical means. Further, known agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-agent binding. Such a reagent may also reduce non-specific or background interactions of the reaction components. Other reagents that improve the efficiency of the assay such as protease inhibitors, nuclease inhibitors, antimicrobial agents and the like may also be used.

The mixture of the foregoing reaction materials is incubated under conditions whereby, the candidate agent interacts with the cysteine-modified PSMA polypeptide, e.g., the dimer thereof. The order of addition of components, incubation temperature, time of incubation and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of e.g., PSMA enzyme activity, is detected by any convenient method available to the user. For example, the reaction mixture can contain a substrate. Preferably the substrate and/or the product formed by the action are detectable. The substrate usually comprises, or is coupled to, a detectable label. A wide variety of labels can be used, such as those that provide direct detection (e.g., radioactivity, luminescence, optical, or electron density, etc) or indirect detection (e.g., epitope tag such as the FLAG epitope, enzyme tag such as horseradish peroxidase, etc.). The label may be bound to the substrate, or incorporated into the structure of the substrate.

A variety of methods may be used to detect the label, depending on the nature of the label and other assay components. For example, the label may be detected while bound to the substrate or subsequent to separation from the substrate. Labels may be directly detected through optical or electron density, radioactive emissions, nonradiative energy transfers, etc. or indirectly detected with antibody conjugates, strepavidin-biotin conjugates, etc. Methods for detecting a variety of labels are well known in the art.

The compositions of the present invention have in vitro and in vivo utilities. For example, these compositions can be administered to cells in culture, e.g., in vitro or ex vivo, or in a subject, e.g., in vivo, to treat, prevent, etc. a variety of disorders. The compositions provided herein can be given to any subject in need thereof. As used herein, the term “subject” is intended to include humans and non-human animals. Preferred subjects include a human patient having a disorder characterized by expression, typically aberrant expression (e.g., overexpression) of PSMA. Other preferred subjects include subjects that are treatable with the compositions of the invention. This includes those who have or are at risk of having a cancer or who would otherwise would benefit from the stimulation of an immune response to cells expressing PSMA. In some embodiments these cells express PSMA on their surface. As another example, the compositions provided can be given to a conventional cancer treatment-experienced patient.

The compositions of the present invention may include or be diluted into a pharmaceutically-acceptable carrier. As used herein, “pharmaceutically acceptable carrier” or “physiologically acceptable carrier” means one or more compatible solid or liquid fillers, diluents or encapsulating substances which are suitable for administration to a human or other mammal such as a primate, dog, cat, horse, cow, sheep, or goat. Such carriers include any and all salts, solvents, dispersion media, coatings, antibacterial and antifungal agents, isotonic and absorption delaying agents, and the like that are physiologically compatible. The term “carrier” denotes an organic or inorganic ingredient, natural or synthetic, with which the active ingredient is combined to facilitate the application. The carriers are capable of being comingled with the preparations of the present invention, and with each other, in a manner such that there is no interaction which would substantially impair the desired pharmaceutical efficacy or stability. Preferably, in some embodiments, the carrier is suitable for oral, intranasal, intravenous, intramuscular, subcutaneous, parenteral, spinal, intradermal or epidermal administration (e.g., by injection or infusion). Suitable carriers can be found in Remington's Pharmaceutical Sciences, Mack Publishing Company, Easton, Pa. Depending on the route of administration, the active compound may be coated in a material to protect the compound from the action of acids and other natural conditions that may inactivate the compound.

When administered, the compositions of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. The components of the pharmaceutical compositions also are capable of being co-mingled in a manner such that there is no interaction which would substantially impair the desired pharmaceutical efficacy. Such preparations may routinely contain salts, buffering agents, preservatives, compatible carriers, and optionally other therapeutic agents, such as supplementary immune potentiating agents including adjuvants, chemokines and cytokines. When used in medicine, the salts should be pharmaceutically acceptable, but non-pharmaceutically acceptable salts may conveniently be used to prepare pharmaceutically-acceptable salts thereof and are not excluded from the scope of the invention.

A salt retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of the invention also may include isotonicity agents. This term is used in the art interchangeably with iso-osmotic agent, and is known as a compound which is added to a pharmaceutical preparation to increase the osmotic pressure to that of 0.9% sodium chloride solution, which is iso-osmotic with human extracellular fluids, such as plasma. Preferred isotonicity agents, in some embodiments, are sodium chloride, mannitol, sorbitol, lactose, dextrose and glycerol.

Optionally, the compositions of the invention may further comprise a preservative, such as benzalkonium chloride. Suitable preservatives also include but are not limited to: chlorobutanol (0.3-0.9% W/V), parabens (0.01-5.0%), thimerosal (0.004-0.2%), benzyl alcohol (0.5-5%), phenol (0.1-1.0%), and the like.

The compositions of the invention may also comprise a diluent. Diluents include water suitable for injection, saline, PBS, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Surfactants as well as other excipients can also be included in the compositions provided herein. Examples of surfactants include those known in the art and described herein. For example, surfactants include Triton X-100, dodecylmaltoside, cholic acid and CHAPS. Examples of excipients include binders, coatings, compression/encapsulation aids, disintegrants, creams and lotions, lubricants, materials for chewable tablets, parenterals, plasticizers, powder lubricants, soft gelatin capsules, spheres for coating, spheronization agents, suspending/gelling agents, sweeteners and wet granulation agents. Specific examples of such excipients include acetyltriethyl citrate (ATEC); acetyltri-n-butyl citrate (ATBC); aspartame; aspartame and lactose; alginates; calcium carbonate; carbopol; carrageenan; cellulose acetate phthalate-based coatings; cellulose-based coatings; cellulose and lactose combinations; colorants for film coating systems; croscarmellose sodium; crospovidone; dextrose; dibutyl sebacate; ethylcellulose-based coatings; fructose; gellan gum; glyceryl behenate; honey; lactose; anhydrous; lactose; monohydrate; lactose and aspartame; lactose and cellulose; lactose and microcrystalline cellulose; L-HPC (Low-substituted HydroxyPryopl Cellulose); magnesium stearate; maltodextrin; maltose DC; mannitol DC; methylcellulose-based coatings; microcrystalline cellulose; methacrylate-based coatings; microcrystalline cellulose and carrageenan; microcrystalline cellulose and guar gum; microcrystalline cellulose and lactose; microcrystalline cellulose and sodium carboxymethylcellulose; molasses DC; polyvinyl acetate phathalate (PVAP); povidone; shellac; sodium starch glycolate; sorbitol, crystalline; sorbitol, special solution; starch DC; sucrose DC; sugar spheres; triacetin; triethylcitrate and xanthan gum. Other excipients include antioxidants and cryoprotectants.

Antioxidants are substances capable of inhibiting oxidation by removing free radicals from solution. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, butylated hydroxy toluene, alkylgallate, dithiothreitol (DTT), sodium meta-bisulfite, sodium bisulfite, sodium dithionite, sodium thioglycollic acid, sodium formaldehyde sulfoxylate, tocopherol and derivatives thereof (e.g., d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, and sodium sulfite. Such materials are typically added in ranges from about 0.01 to about 2%.

The compositions provided can be lyophilized. For a lyophilized product or a product stored in the cold, one or more cryoprotectants can be added, and such compositions are also provided. Typical cryoprotectants for polypeptides include but are not limited to: sugars such as sucrose, lactose, glucose, trehalose, maltose, and the like; polyols such as inositol, ethylene glycol, glycerol, sorbitol, xylitol, mannitol, 2-methyl-2,4-pentane-diol and the like; amino acids such as Na glutamate, proline, alpha-alanine, beta-alanine, glycine, lysine-HCl, 4-hydroxyproline; polymers such as polyethylene glycol, dextran, polyvinylpyrrolidone and the like; inorganics salts such as sodium sulfate, ammonium sulfate, potassium phosphate, magnesium sulfate, and sodium fluoride and the like; organics salts such as sodium acetate, sodium polyethylene, sodium caprylate, proprionate, lactate, succinate and the like; as well as agents such as trimethylamine N-oxide, sarcosine, betaine, gamma-aminobutyric acid, octapine, alanopine, strombine, dimethylsulfoxide, and ethanol.

The compositions provided herein also include those that are sterile. Sterilization processes or techniques as used herein include aseptic techniques such as one or more filtration (0.45 or 0.22 micron filters) steps.

The compositions provided may contain suitable buffering agents, including: acetic acid in a salt; citric acid in a salt; boric acid in a salt; and phosphoric acid in a salt.

The compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administration can be found in Remington's Pharmaceutical Sciences, Mack Publishing Co., Easton, Pa.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The compositions of the invention can be administered by any conventional route, including injection or by gradual infusion over time. The administration may, for example, be oral, subcutaneous, intravenous, intraperitoneal, intramuscular, intracavity, intratumor, or transdermal. In some embodiments subcutaneous or intramuscular administration is preferred. Routes of administration also include by pulmonary aerosol. Techniques for preparing aerosol delivery systems containing a therapeutic are well known to those of skill in the art. Generally, such systems should utilize components which will not significantly impair the biological properties of a compound (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing aerosols without resorting to undue experimentation.

The compositions of the invention, when used in alone or in combination with other therapeutics (e.g., in cocktails), are administered in therapeutically effective amounts. Effective amounts are well known to those of ordinary skill in the art and are described in the literature. A therapeutically effective amount will be determined by the parameters discussed below; but, in any event, is that amount which establishes a level of a therapeutic or combination of therapeutics effective for treating a subject, such as a human subject, having one of the conditions described herein. An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

More specifically, an “effective amount” is that amount of the compositions provided that alone, or together with further doses and/or other therapeutic treatments, produces the desired response, e.g., stimulates an immune response, treats cancer in a subject, etc. The term is also meant to encompass the amount of the compositions that in combination with one or more other therapeutic agents/treatment regimens produce the desired response. This may involve only slowing the progression of the disease temporarily, although more preferably, it involves halting the progression of the disease permanently. This can be monitored by routine methods. The desired response to treatment of the disease or condition also can be delaying the onset or even preventing the onset of the disease or condition.

The doses of the compositions administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

A variety of administration routes are available. The particular mode selected will depend of course, upon the particular therapeutic selected, the severity of the disease state being treated and the dosage required for therapeutic efficacy. The methods of this invention, generally speaking, may be practiced using any mode of administration that is medically acceptable, meaning any mode that produces effective levels of the therapeutics without causing clinically unacceptable adverse effects. Such modes of administration include oral, rectal, sublingual, topical, nasal, transdermal or parenteral routes. The term “parenteral” includes subcutaneous, intravenous, intramuscular or infusion.

In general, doses can range from about 50 μg to about 100,000 mg. In one embodiment the dose is about 50 μg-1 mg. In another embodiment the dose is about 1-5 mg. In still another embodiment the dose is about 5-10 mg. In another embodiment the dose is about 10-100 mg. In yet another embodiment the dose is about 100-1000 mg. In still another embodiment the dose is about 0.5 mg (e.g., when the composition is a polypeptide vaccine composition). In another embodiment the dose is about 300 mg. In still another embodiment the dose is about 500 mg, 1000 mg or greater. Based upon the composition, the dose can be delivered once, continuously, such as by continuous pump, or at periodic intervals. The periodic interval may be weekly, bi-weekly or monthly. The dosing can occur over a period of one month, two months, three months or more to, for example, elicit an appropriate humoral and/or cellular immune response. Desired time intervals of multiple doses of a particular composition can be determined without undue experimentation by one skilled in the art. Other protocols for administration will be known to one of ordinary skill in the art, in which the dose amount, schedule of administration, sites of administration, mode of administration and the like vary from the foregoing.

Dosage may be adjusted appropriately to achieve desired drug levels, locally or systemically. Generally, daily oral doses of active compounds will be from about 0.1 mg/kg per day to 30 mg/kg per day. It is expected that IV doses in the range of 0.01-1.00 mg/kg will be effective. In the event that the response in a subject is insufficient at such doses, even higher doses (or effective higher doses by a different, more localized delivery route) may be employed to the extent that patient tolerance permits. Continuous IV dosing over, for example, 24 hours or multiple doses per day also are contemplated to achieve appropriate systemic levels of compounds.

Administration of the compositions to mammals other than humans, e.g., for testing purposes or veterinary therapeutic purposes, is carried out under substantially the same conditions as described above.

It should be understood that the compositions provided will typically be held in bottles, vials, ampoules, infusion bags, and the like, any one of which may be sparged to eliminate oxygen or purged with nitrogen. In some embodiments, the bottles vials and ampoules are opaque, such as when amber in color. Such sparging and purging protocols are well known to those of ordinary skill in the art and should contribute to maintaining the stability of the compositions. The compositions also, in certain embodiments, are expected to be contained within syringes.

Also provided are kits comprising the compositions provided herein. The kits provided include any of the compositions described and instructions for the use of these compositions. The instructions can include instructions for mixing a particular amount of a polypeptide or nucleic acid composition provided with a particular amount of an additional reagent, such as an additional therapeutic, adjuvant, cytokine, etc. The instructions can also include instructions for mixing a particular amount of a diluent with a particular amount of a polypeptide or nucleic acid composition, whereby a final formulation for injection or infusion is prepared. Therefore, kits are also provided, which include the compositions of the invention and, optionally, an adjuvant (e.g., alum) or diluent and instructions for mixing. Kits are also provided wherein the compositions of the inventions are provided in a vial or ampoule with a septum or a syringe. The instructions, therefore, would take a variety of forms depending on the presence or absence of diluent or other reagents (e.g., therapeutics). The instructions can include instructions for treating a patient with an effective amount of a composition as provided herein. It also will be understood that the containers containing the compostions, whether the container is a bottle, a vial with a septum, an ampoule with a septum, an infusion bag, and the like, can contain indicia such as conventional markings which change color when the composition has been autoclaved or otherwise sterilized. The components of the kits can be packaged either in aqueous medium or in lyophilized form. Kits for use in in vivo therapy containing the compositions provided can be prepared.

When the polypeptides or nucleic acids are used in kits with other reagents, the components can be supplied either in separate containers, the contents of which can be mixed by the user of the kit, or as a mixture in a single container. A kit may comprise a carrier being compartmentalized to receive in close confinement therein one or more container means or series of container means such as test tubes, vials, flasks, bottles, syringes or the like. A first of said container means or series of container means may contain one or more of the compositions provided. A second container means or series of container means may contain an additional reagent.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Methods and Materials

### Constructs

The cDNA encoding the rsPSMA gene was PCR amplified using the eukaryotic expression vector (pPI4/dhfr/rsPSMA) as template DNA. The forward and the reverse PCR primers used in the amplification were designed to contain a Hind III restriction site at the 5′ end (forward primer) and Sma 1 restriction site at the 3′ end (reverse primer), respectively. Subsequently, the PCR amplified rsPSMA gene was digested with Hind III and Sma I restriction enzymes and cloned into the pEE14.4 vector (Lonza Biologics plc, Slough, Great Britain) cut with the same enzymes. The generic design of the PCR fragment with Hind III and Sma 1 restriction sites offers flexibility to clone the rsPSMA gene into other more commonly used eukaryotic expression vectors like pcDNA3.1 (Invitrogen, Carlsbad, Calif.; Cat. #V790-20) and pCI vector (Promega; Cat. #E1731).

A nucleic acid encoding rsPSMA can also be obtained by synthesizing the sequence or by PCR amplification

### Mutagenesis

Mutations were performed on the pEE14.4 vector (Lonza Biologics plc) containing the rsPSMA gene insert. The pEE14.4\rsPSMA sample which served as a template for mutagenesis was digested with HindIII and EcoR1 enzymes prior to mutagenesis reactions to confirm the presence of the rsPSMA insert. Mutations were then performed using the QuikChange II XL Site Directed Mutagenesis Kit (Invitrogen, Carlsbad, Calif.) and followed IV the procedure described in the kit manual. Forward and reverse primers containing the desired mutation or insertion used in the mutagenesis reactions were obtained from GeneLink (Hawthorne, N.Y.). Once E. coli host colonies, which were expected to contain mutated copies of the rsPSMA gene, were obtained several colonies for each mutation were selected and inoculated into 5 ml of Luria-Bertani (LB) media containing ampicillin. These E. coli cultures were shaken at 225 rpm and 37° C. for 16 hours. The transformed E. coli were then subjected to a PCR-based diagnostic test to determine whether the desired mutation had been introduced into the parental plasmid vector (pEE14.4\rsPSMA).

The diagnostic test involved performing a PCR reaction using the transformed E. coli as the source of template DNA in the PCR. The forward primer used in each PCR reaction was complimentary to the parental plasmid at the 5′ end and was also complimentary to the desired mutation at the 3′ end such that if the desired mutation was not present then the forward primer would not anneal properly to the parental DNA, and no PCR product would result from the reaction. If, however, the desired mutation was present, the primer would anneal properly, and a PCR product would amplify. The resulting PCR samples were run on a 0.8% agarose pre-caste E-Gel (Invitrogen) with a 1 kb size marker according to the manufacturer's specifications. A photograph was taken of the gel to confirm the PCR fragment of the desired length had been attained. The expected length of PCR fragments from reactions, intended to identify the presence of different mutations, varied according to the locations of the primers used.

pEE14.4\rsPSMA mutated plasmids chosen by diagnostic test were harvested from E. coli hosts using the QIAprep Spin Miniprep Kit (Qiagen, Valencia, Calif.) according to the manufacturer's specifications. Mutated pEE14.4\rsPSMA plasmids were digested with HindIII and EcoR1 to confirm the presence of the intact Lonza pEE14.4 vector and the rsPSMA gene insert. If the mutated plasmids demonstrated the presence of these two components, their respective E. coli host samples were inoculated into 100 ml of LB media containing ampicillin. The transformed E coli were allowed to spin at 225 rpm and 37° C. for 16 hours. Once this culture of transformed E. coli was obtained, the plasmids were harvested using the Hi Speed Plasmid Midiprep Kit (Qiagen) following the manufacturer's specifications. Plasmids obtained from this procedure underwent an additional ethanol precipitation step and were resuspended in a lower volume than recommended by the kit instructions. These additional steps were performed in order to prepare the plasmids for use in transient transfections of 293T cells (ATCC Accession No. CRL-1573). The concentrations of the plasmids in solution were measured directly using a spectrophotometer at an absorbance of A₂₆₀.

### Transfections

The mutated pEE14.4\rsPSMA plasmids were expressed transiently in 293T cells using Lipofectamine 2000 Reagent (Invitrogen) and following the manufacturer's suggested protocol. The recommended quantities from the protocol intended for transfections in 24 well plates were multiplied by a factor of 5 to fit the greater surface area of 6 well plates used in these transfections. Media was changed 4-6 hours after transfections to expression media which did not contain any serum, but in some cases did contain dextran sulfate or polyethylene glycol (PEG) 8000. Expression media was harvested 3-4 days after transfection and was centrifuged at 3000 rpm for 20 minutes to pellet cell debris. The supernatant was removed and stored at 4° C.

### Blots

Expression media harvested from transient transfections were run on 4-12% BisTris NuPAGE gels (Invitrogen). Each sample contained the appropriate volume of 4× NuPAGE LDS loading buffer (Invitrogen) and was heated at 700 C for 10-20 minutes before loading onto a gel. Samples run under reducing conditions contained 10% dithiothreitol (DTT) in addition to LDS loading buffer. In general, protein samples were run alongside a SeeBlue Pre-Stained Standard (Invitrogen) size marker. Gels were run using the Xcell Surelock Mini-Cell (Invitrogen) gel running system with NuPAGE MES SDS Running Buffer (Invitrogen) at 150V for 1 hour. Transfer onto nitrocellulose membrane was performed using Trans-Blot SD Semi-Dry Transfer Cell (Bio-Rad, Hercules, Calif.). Transfer was performed at 25V for 1 hour using NuPAGE MES Transfer Buffer (Invitrogen) containing 20% methanol. After incubating the nitrocellulose membrane in blocking buffer (PBS 5% dry milk 0.5% Tween) overnight, the membrane was probed with mAb544P (Maine Biotech, Portland, Me.), which recognizes a linear epitope of rsPSMA, at a concentration of 1 μg/ml in blocking buffer for 1 hour. Following primary antibody probing, the membrane was washed three times with PBS 0.5% Tween for 15 minutes each. Next, the membrane was incubated with goat anti-mouse IgG horseradish peroxidase (HRP) at 1 μg/ml in blocking buffer for 1 hour. The membrane was washed three times with PBS 0.5% Tween for 15 minutes each and then once in PBS for 15 minutes. The membrane was then incubated with Western Lightning Chemiluminescence Reagent (PerkinElmer, Wellesley, Mass.) according to the manufacturer's specifications. The membrane was then sandwiched between two transparencies, exposed to film, and the film was developed. In order to determine whether mutated protein was similar in conformation to the native protein, its reactivity with a human anti-PSMA monoclonal antibody (anti-PSMA hmAb 006), was determined in an immunoprecipitation procedure using Seize Classic (G) Immunoprecipitation Kit (Pierce, Rockford, Ill.) following the manufacturer's specification. Anti-PSMA hmAb 006 as well as methods of making the monoclonal antibody are disclosed in WO 03/34903. The description of the antibody and methods of its production are expressly incorporated by reference herein. Anti-PSMA hmAb 006 specifically recognizes dimeric but not monomeric rsPSMA. In addition, anti-PSMA hmAb 006 efficiently binds PSMA-expressing tumor cells, but not denatured PSMA, and thus defines an epitope unique to the quaternary structure of PSMA. Plasmids encoding the heavy and light chains of anti-PSMA hmAb 006 are deposited with the American Type Culture Collection (ATCC) (PTA-4403 and PTA-4404, respectively).

The Bio-Rad Dot Blot Apparatus (Bio-Rad) was used to blot proteins to nitrocellulose membranes in order to detect reactive proteins with anti-PSMA hmAb 006. This procedure has the advantage over conventional Western blotting in that it allows proteins of interest to be detected in a native conformation (without denaturation by detergents or boiling). The first step was to prepare a nitrocellulose membrane (Bio-Rad; Cat. #162-0148) by cutting to correct size (10 cm×8 cm) and notching the bottom-right corner of the membrane in order to be able to identify the corresponding wells of a 96 well plate. The membrane was then wetted by soaking in wash buffer (PBS w/o Ca, Mg (Invitrogen; Cat. #14190-136) with 0.5% w/v of Tween 20 (Sigma, St. Louis, Mo.; Cat. #P7949)). The wetted membrane was then transferred to the Dot Blot Apparatus and placed on top of the gasket seal such that the notched corner of the membrane was at the bottom-right corner of the apparatus. The cover of the apparatus was then screwed down hand-tight. The protein samples (0.1-1.0 μg of protein in a volume of 100 μl) were placed into the wells according to a 96 well plate index. A range of purified standards along with wells containing blank media or diluent were used to determine background reactivity and approximate titer of protein of interest.

After all of the samples were placed in the wells, a vacuum line was attached to the Dot Blot Apparatus. Vacuum was applied in order to suck the samples through the wells and transfer the protein to the membrane. The membrane was then removed from the Dot Blot apparatus and washed three times in wash buffer to remove excess unbound protein. The membrane was then blocked in blocking buffer (wash buffer with 5% instant milk (Carnation Non-Fat Dry Milk)) for 1 hour. The membrane was then washed again three times in washing buffer to removes excess milk. The membrane was probed with anti-PSMA hmAb 006 as per Western blot protocol (one hour incubation followed by three 10 minute washes in wash buffer). An additional wash in PBS without Ca, Mg or Tween was done before blots were developed by detection of HRP-conjugated secondary antibody, goat anti-human IgG, binding to membrane using Western Lightning chemiluminescent reagent (Perkin-Elmer; Cat. #NEL 102). Membranes were soaked in the chemiluminescent reagent for 2 minutes and then placed between two sheets of transparencies in a film cassette. Blots were developed by exposing to light sensitive film (Kodak X-AR film) for 30 seconds to 5 minutes depending on the intensity of the signal.

### Results

Sites in the helical and stalk domain were selected as likely to cause disulfide bond formation with their counterparts in other rsPSMA monomers when mutated to cysteines. However, mutations in the helical domain, such as mutation of 6041, which was noted as being the most promising site for disulfide-bond-forming cysteine substitution, was observed to cause conformational changes to the rsPSMA protein when mutated resulting in insoluble protein, non-reactive with anti-PSMA hmAb 006 (see Table 1). Residues 3S, 5E, 6A and 7T of the stalk region of the rsPSMA protein were mutated to cysteines. In addition, a four amino acid insertion from the constant region of a human IgG (tgcccaccgtgc (SEQ ID NO: 13)) was placed between the first and second amino acids of the stalk region of the protein. One of ordinary skill in the art will recognize that degenerate versions of this sequence can also be used. The substitutions and insertion in the stalk region each resulted in the production of soluble active dimeric protein. The substitutions and insertion were shown to not affect the general structure of rsPSMA when mutated as evidenced by anti-PSMA hmAb 006 recognition of the mutant dimeric proteins.

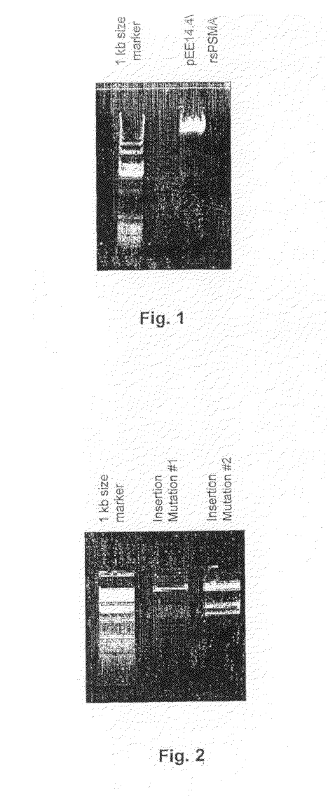

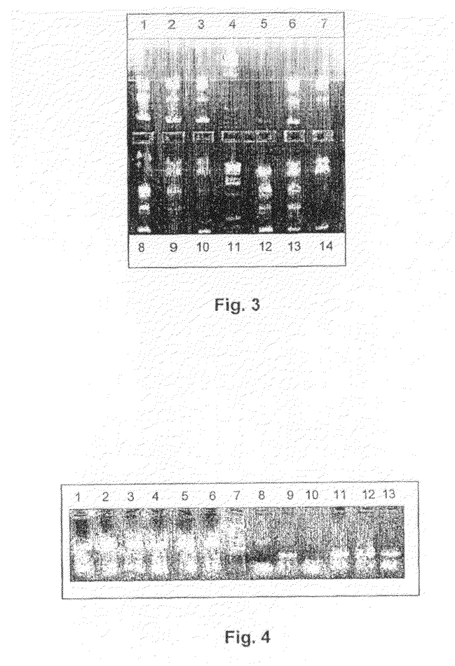

Restriction digestion analysis of pEE14.4\rsPSMA with HindIII and EcoR1 demonstrated the expected 0.8 kb and 1.3 kb bands according to the location of the digestion sites in the pEE14.4\rsPSMA plasmid (FIG. 1). Later restriction digestion analysis of pEE14.4\rsPSMA mutants also revealed the presence of the same expected bands (FIG. 2).

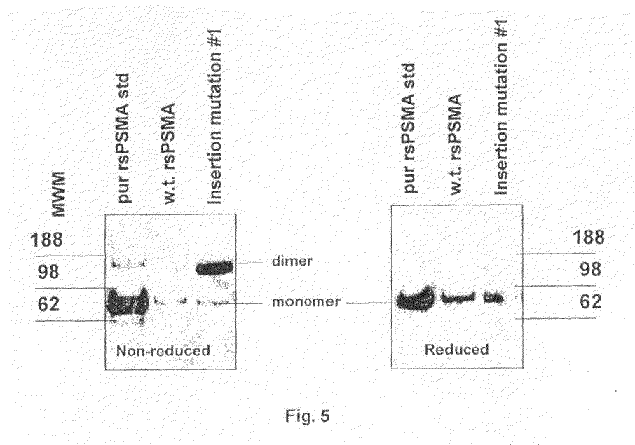

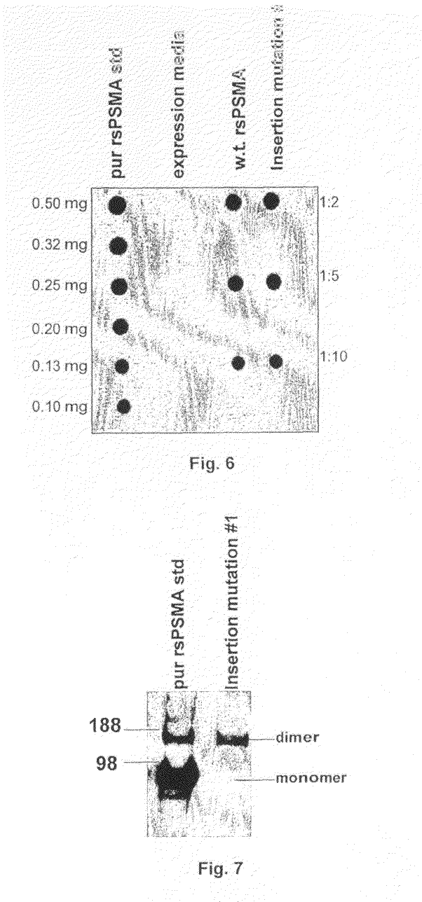

S PCR-based diagnostic tests for the four amino acid insertion mutation were expected to produce PCR bands of approximately 300 bp based on the location of the forward and reverse primers. Though most samples tested demonstrated the presence of this band, negative results were also obtained (FIG. 3). Similarly, PCR-based diagnostic tests for 623P-C mutation, expected to produce bands approximately 250 bp in length, also indicated that most samples seemed to contain the desired mutation (FIG. 4). Alternately, a DNA band resulting from a non-specific PCR reaction was observed in the PCR reactions testing for the presence of the 389E-C mutation. Nevertheless, the band of the desired length according to the location of the primers, approximately 850 bp, was still observed to be present in some samples despite the occurrence of a non-specific reaction (FIG. 4).

Though several samples exhibited PCR fragments of desired lengths, only 2 clones of the insertion mutant (termed insertion mutant #1 and insertion mutant #2) and 1 clone each from the 389E-C and 623P-C mutations were selected for plasmid preparation. Once plasmid preparation was completed for all the selected pEE14.4\rsPSMA mutated samples and also a non-mutated pEE14.4\rsPSMA sample, a spectrophotometer was used to determine the concentration of the plasmids in solution. These concentrations were found to be 2.2 μg/μl for the non-mutated pEE14.4\rsPSMA plasmid, 1.5 μg/μl for pEE14.4\rsPSMA plasmid with insertion mutation #1, 2.0 μg/μl for pEE14.4\rsPSMA plasmid with insertion mutation #2, and 0.4 μg/μl for both the pEE14.4\rsPSMA plasmid with 389E-C mutation and the pEE14.4\rsPSMA plasmid with 623P-C mutation.

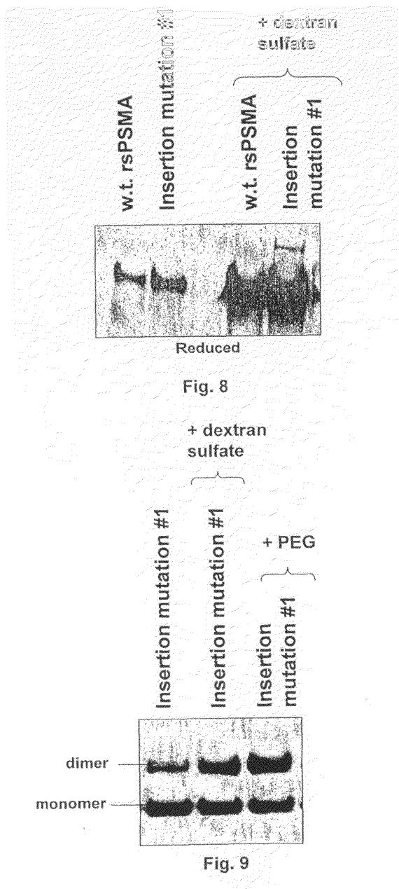

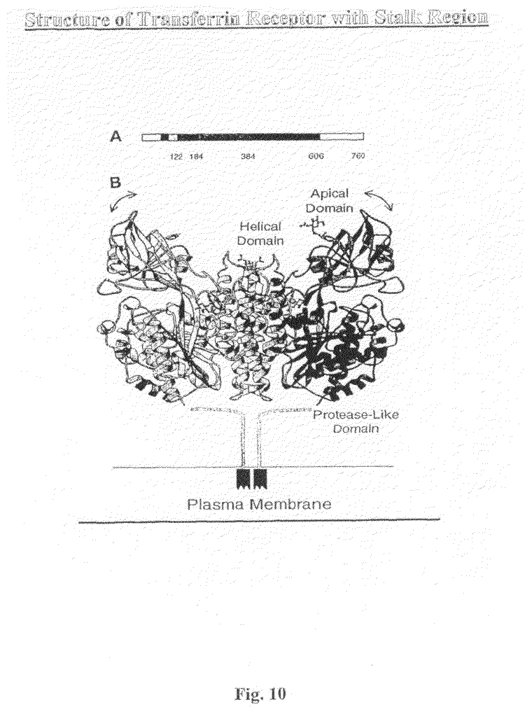

Under denaturing conditions wild type rsPSMA from transient transfections, as well as purified rsPSMA protein, both appeared on Western blots in monomer configuration as expected given the absence of an intersubunit disulfide-bond. A portion of rsPSMA stalk region insertion mutant #1 retained its dimer form under denaturing conditions (FIG. 5). The pEE14.4\rsPSMA insertion mutant #2 and the 389E-C and 623P-C mutations failed to express at high enough levels to be detected by a Western blot. In dot blots probed with anti-PSMA hmAb 006, which recognizes rsPSMA dimer, the insertion mutant #1 was found to be reactive, indicating that this mutation led to the production of protein in a native conformation (FIG. 6). However, insertion mutant #2, 389E-C and 623P-C rsPSMA mutants were not detected by dot blot.

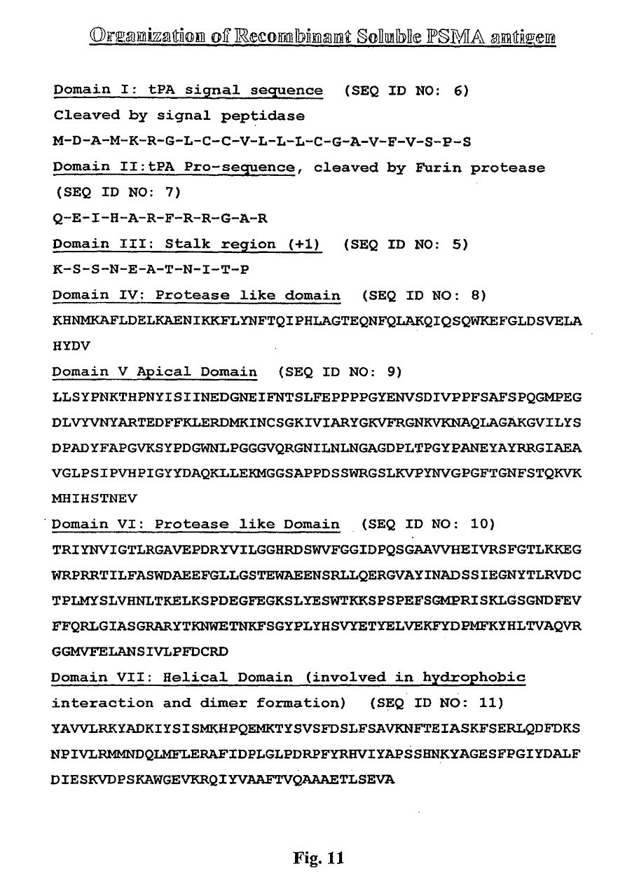

Western blots were also performed using rsPSMA insertion mutant #1 samples immunoprecipitated from the expression media using anti-PSMA hmAb 006, which only recognizes rsPSMA dimer. When run on denaturing gels, these samples of rsPSMA insertion mutant #1 appeared almost entirely in dimer configuration (FIG. 7), indicating that the disulfide-bond was formed efficiently.

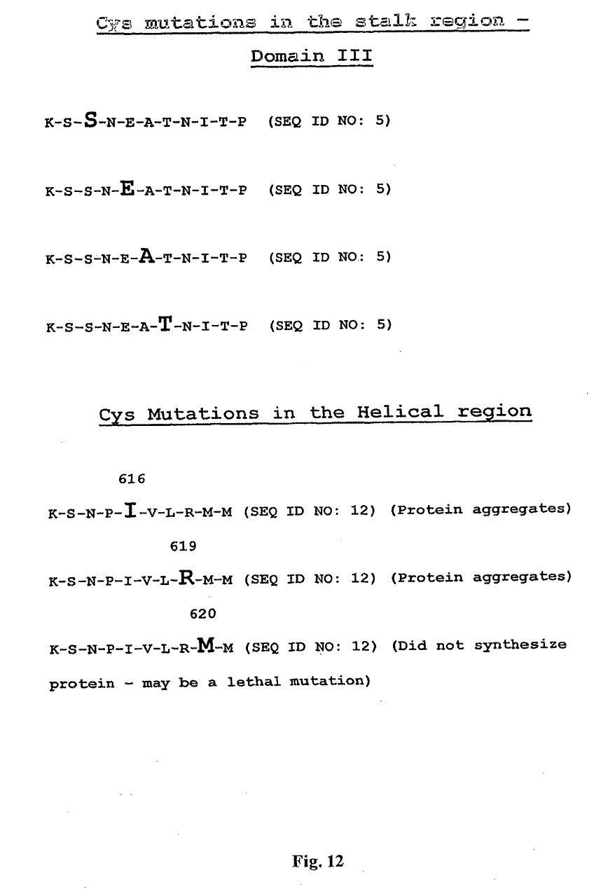

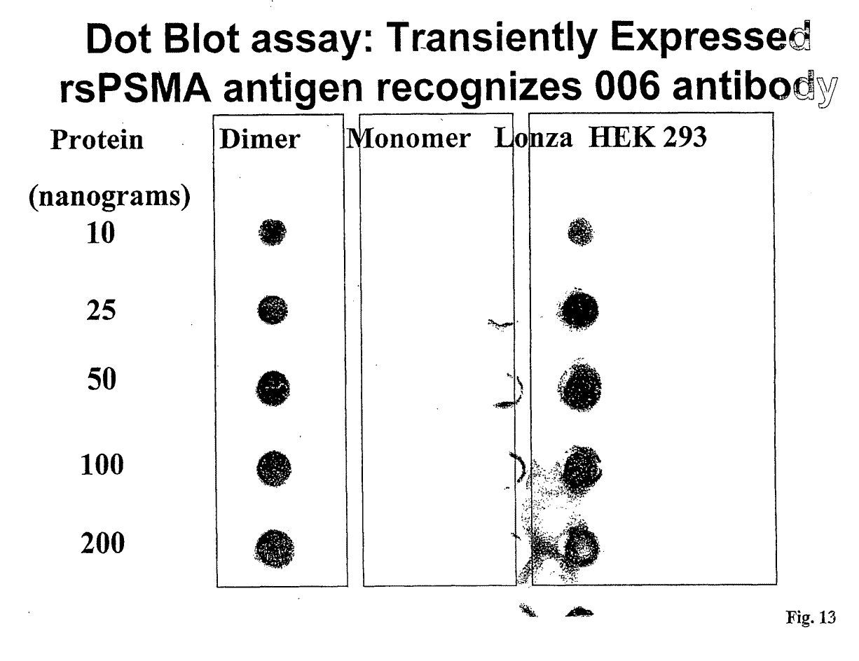

The effects of introducing dextran sulfate and PEG into the 293T expression media were gauged using Western blots from denaturing gels. Cells transfected with wild type pEE14.4\rsPSMA and pEE14.4\rsPSMA insertion mutant #1 with expression media containing dextran sulfate were observed to express at higher levels (FIG. 8). Furthermore, the presence of PEG in the expression media appeared to raise slightly the dimer to monomer ratio (FIG. 9).

### Discussion

The pEE14.4\rsPSMA insertion mutant #1 consistently expressed detectable levels of protein over the course of several transfections. The majority of this rsPSMA mutant did retain its dimer configuration under denaturing conditions, indicating that a cysteine-mediated covalent bond existed between the monomer peptides of the mutant. Not all mutated protein was found to be in stable dimer form, and monomer bands were still visible on Western blots performed under denaturing conditions.

In order to attain a more favorable dimer to monomer ratio, PEG 8000 was introduced into the expression media of rsPSMA insertion mutant #1. PEG 8000 is a compound which is used to potentiate hydrophobic interactions of proteins. It seems the presence of PEG 8000 in the expression media caused a slight improvement in the dimer to monomer ratio of insertion mutant #1. In addition, the use of dextran sulfate as a component of the expression media seemed to improve expression of both wild type rsPSMA and the rsPSMA insertion mutant. This is probably due to dextran sulfate's ability to extend cell life thereby prolonging the time during which 293T cells expressed proteins.

To confirm that rsPSMA insertion mutant #1 retains rsPSMA's native conformation, dot blots testing insertion mutant #1 for reactivity with anti-PSMA hmAb 006 were performed. Positive results for reactivity in these dot blots indicated that rsPSMA insertion mutant #1 retained the native conformation of rsPSMA. Also, the selection of rsPSMA insertion mutant #1 stable dimer by immunoprecipitation using anti-PSMA hmAb 006 indicated specifically that the disulfide-bond-mediated dimer engineered retains rsPSMA's native conformation. The results for the rsPSMA mutants created are shown in Table 1.

TABLE 1
Summary Table

| | Cys | Active | | Insoluble | Binding to |
|---|---|---|---|---|---|
| Domain | substitution | Dimer | Monomer | Protein | 006 |
| Helical | 620 Ile | − | − | + | − |
| | 623 Arg | − | − | + | − |
| | 625 Met | − | − | + | − |
| | 639 Pro | − | − | + | − |
| Stalk | 3 Ser | ++ | + | − | + |
| | 5 Glu | ++ | + | − | + |
| | 6 Ala | + | + | − | + |
| | 7 Thr | +/− | + | − | + |
| Insertion | C-P-P-C | ++++ | + | − | ++ |
| | (IgG1 Hinge) | | | | |

### CONCLUSION

rsPSMA mutants, expressed transiently in stable dimer configuration as a result of a cysteine-mediated covalent link between its monomer components, were successfully engineered. In addition, it was demonstrated that the conformation of native PSMA (recognized by anti-PSMA hmAb 006) was retained in the rsPSMA mutants containing cysteine substitutions or the cysteine-containing insertion sequence in the stalk region. Furthermore, it was found that the addition of anti-apoptotic agents and/or PEG to the expression media is useful in process development to optimize the amount and concentration of stable dimer produced.

### REFERENCES

1. Dhanasekaran, S. M., Barrette, T. R., Ghosh, D., Shah, R., Varambally, S., Kurachi, K., Pienta, K. J., Rubin, M. A. & Chinnaiyan, A. M. (2001) Nature 412, 822-826.
2. Norbert Schülke, Olga A, Varlamova, Gerald P. Donovan, Dangshe Ma, Jason P Gardner, Donna M. Morrissey, Robert R. Arrigale, Cenchen Zhan, Amy J. Chodera, Kenneth G. Surowitz, Paul J. Maddon, Warren D. W. Heston, and William C. Olson (2003) PNAS 100, 12590-12595.

Although the invention has been described in detail for the purpose of illustration, it is understood that such detail is solely for that purpose and variations can be made by those skilled in the art without departing from the spirit and scope of the invention which is defined by the following claims.

The contents of all references, patents and published patent applications cited throughout this application are Incorporated herein by reference.

The citation of a reference herein is not intended to be an admission that the reference is a prior art reference.

1. A cysteine-modified PSMA polypeptide, comprising:
a cysteine-modified stalk region, and
an amino acid sequence set forth as SEQ ID NO: 4 or a fragment thereof.

2. The cysteine-modified PSMA polypeptide of claim 1, wherein the cysteine-modified stalk region has an amino acid sequence as set forth in SEQ ID NO: 5 except that one or more residues of SEQ ID NO: 5 are substituted with cysteine.

3. The cysteine-modified PSMA polypeptide of claim 2, wherein one, two or three residues of SEQ ID NO: 5 are substituted with cysteine.

4. The cysteine-modified PSMA polypeptide of claim 3, wherein one of the residues substituted with cysteine corresponds to the residue at position 1, 2, 3, 4, 5, 6 or 7 of SEQ ID NO: 5.

5. The cysteine-modified PSMA polypeptide of claim 4, wherein one of the residues substituted with cysteine corresponds to the residue at position 1, 2, 3, 4 or 5 of SEQ ID NO: 5.

6. The cysteine-modified PSMA polypeptide of claim 5, wherein one of the residues substituted with cysteine corresponds to the residue at position 1, 2 or 3 of SEQ ID NO: 5.

7. The cysteine-modified PSMA polypeptide of claim 6, wherein one of the residues substituted with cysteine corresponds to the residue at position 3 of SEQ ID NO: 5.

8. The cysteine-modified PSMA polypeptide of any of claims 3-7, wherein one residue of SEQ ID NO: 5 is substituted with cysteine.

9. The cysteine-modified PSMA polypeptide of claim 1, wherein the cysteine-modified stalk region has an amino acid sequence as set forth in SEQ ID NO: 5 except that one of the residues at position 2, 3, 4, 5, 6, 7, 8, 9, 10 or 11 of SEQ ID NO: 5 is substituted with cysteine and the residue at position 1 of SEQ ID NO: 5 is substituted with a non-positively charged amino acid.

10. The cysteine-modified PSMA polypeptide of claim 9, wherein the non-positively charged amino acid is cysteine, glycine, alanine, glutamine, glutamic acid, aspartic acid or asparagine.

11. The cysteine-modified PSMA polypeptide of claim 1, wherein the cysteine-modified stalk region has the amino acid sequence as set forth in SEQ ID NO: 5 except that one or more cysteine residues are inserted therein.

12. The cysteine-modified PSMA polypeptide of claim 11, wherein the one or more cysteine residues are inserted after the residue that corresponds to the residue at position 1 of SEQ ID NO: 5.

13. The cysteine-modified PSMA polypeptide of claim 12, wherein two cysteine residues are inserted after the residue that corresponds to the residue at position 1 of SEQ ID NO: 5.

14. The cysteine-modified PSMA polypeptide of claim 11, wherein the one or more cysteine residues are part of an amino acid sequence, X¹ₙ-X²-X³-X⁴-X⁵-X⁶ₙ, and it is the amino acid sequence that is inserted, and wherein n is 0 or 1.

15. The cysteine-modified PSMA polypeptide of claim 14, wherein the amino acid sequence contains at least two, three or four cysteines.

16. The cysteine-modified PSMA polypeptide of claim 11, wherein the one or more cysteine residues are part of the amino acid sequence, C-X¹ₙ-X²ₙ-C, and it is the amino acid sequence that is inserted, wherein X¹ and X² are each any amino acid residue and n is 0, 1 or 2.

17. The cysteine-modified PSMA polypeptide of claim 16, wherein n is 1.

18. The cysteine-modified PSMA polypeptide of claim 16, wherein X¹ and X² are each proline or serine.

19. The cysteine-modified PSMA polypeptide of claim 18, wherein X¹ and X² are each proline.

20. The cysteine-modified PSMA polypeptide of claim 18, wherein X¹ is proline and X² is serine.

21. The cysteine-modified PSMA polypeptide of claim 1, consisting of the cysteine-modified stalk region and the amino acid sequence set forth as SEQ ID NO: 4.

22. A composition, comprising:
the cysteine-modified PSMA polypeptide of claim 1.

23. A composition, comprising:
a disulfide-bond-stabilized PSMA dimer, which is formed from two cysteine-modified PSMA polypeptides, each of which is a cysteine-modified PSMA polypeptide of claim 1.

24. The composition of claim 22 or 23, wherein the composition further comprises an adjuvant.

25. The composition of claim 22 or 23, wherein the composition further comprises an additional therapeutic agent.

26. The composition of claim 25, wherein the therapeutic agent is docetaxel.

27. The composition of claim 26, wherein the composition further comprises prednisone.

28. The composition of claim 22 or 23, wherein the composition further comprises a cytokine.

29. The composition of claim 22 or 23, wherein the composition further comprises a pharmaceutically acceptable carrier.

30. The composition of claim 22 or 23, wherein the composition is sterile.

31. The composition of claim 22 or 23, wherein the composition is physiologically acceptable.

32. The composition of claim 22 or 23, wherein the composition is in a liquid or lyophilized form.

33. A nucleic acid molecule that encodes the cysteine-modified PSMA polypeptide of claim 1.

34. The nucleic acid of claim 33, wherein the nucleic acid is DNA or RNA.

35. A vector comprising the nucleic acid molecule of claim 33 operably linked to a promoter.

36. The vector of claim 35, wherein the vector is a plasmid or viral vector.

37. The vector of claim 36, wherein the vector is a DNA plasmid.

38. The vector of claim 36, wherein the viral vector is a pox virus, a herpes virus, adenovirus, vaccinia virus or alphavirus vector.

39. A host cell transformed or transfected with the vector of claim 35.

40. A composition, comprising:
the nucleic acid of claim 33.

41. A composition, comprising:
the vector of claim 35.

42. A composition, comprising:
the host cell of claim 39.

43. The composition of any of claims 40-42, wherein the composition further comprises an adjuvant.

44. The composition of any of claims 40-42, wherein the composition further comprises an additional therapeutic agent.

45. The composition of any of claims 40-42, wherein the composition further comprises a cytokine.

46. The composition of any of claims 40-42, wherein the composition further comprises a pharmaceutically acceptable carrier.

47. The composition of any of claims 40-42, wherein the composition is sterile.

48. The composition of any of claims 40-42, wherein the composition is physiologically acceptable.

49. A method of stimulating an immune response, comprising:
administering the composition of claim 22 or 33 to a subject in an amount effective to stimulate an immune response.

50. (canceled)

51. The method of claim 49, wherein the method further comprises administering one or more booster doses of a composition comprising a PSMA polypeptide or a nucleic acid molecule encoding a PSMA polypeptide.

52. The method of claim 51, wherein the booster dose composition is a PSMA polypeptide.

53. The method of claim 52, wherein the booster dose composition comprises a cysteine-modified PSMA polypeptide comprising a cysteine-modified stalk region, and an amino acid sequence set forth as SEQ ID NO:4 or a fragment thereof.

54. The method of claim 51, wherein the booster dose composition is a nucleic acid molecule encoding a PSMA polypeptide.

55. The method of claim 54, wherein the booster dose composition comprises a nucleic acid molecule that encodes a cysteine-modified PSMA polypeptide comprising a cysteine-modified stalk region, and an amino acid sequence set forth as SEQ ID NO:4 or a fragment thereof.

56. The method of claim 49, wherein the immune response is an immune response to cells in the subject that express PSMA.

57. The method of claim 49, wherein the composition is administered by intravenous, intramuscular, subcutaneous, parenteral, spinal, intradermal or epidermal administration.

58. The method of claim 57, wherein the composition is administered by subcutaneous or intramuscular administration.

59. The method of claim 49, wherein the subject has or has been treated for cancer.

60. The method of claim 49, wherein the subject has or has been treated for prostate cancer.

61. The method of claim 49, wherein the method further comprises harvesting antibodies produced as a result of the immune response.

62. A method of treating cancer in a subject, comprising:
administering to the subject a therapeutically effective amount of the composition of any of claims 22, 23 and 40-42, wherein the composition is effective in treating cancer.

63. The method of claim 62, wherein the cancer is prostate cancer.

64. The method of claim 63, wherein the method further comprises administering to the subject a conventional prostate cancer therapy.

65. The method of claim 64, wherein the conventional prostate cancer therapy is surgery, radiation, cryosurgery, thermotherapy, hormone therapy or chemotherapy.

66. A kit which comprises the composition of any of claims 22, 23 and 40-42 and instructions for use.

67-70. (canceled)

71. A method of producing a PSMA polypeptide, comprising:
modifying a nucleic acid molecule that encodes a PSMA polypeptide comprising the stalk region of PSMA so that the nucleic acid molecule codes for a cysteine residue within the stalk region, and
transfecting cells with a vector containing the modified nucleic acid molecule.

72. The method of claim 71, wherein the nucleic acid molecule is modified to code for a cysteine substitution within the stalk region.

73. The method of claim 71, wherein the nucleic acid molecule is modified to code for a cysteine insertion within the stalk region.

74. The method of claim 71, wherein the method further comprises harvesting and purifying PSMA polypeptide expressed by the transfected cells.

75. The method of claim 71, wherein the PSMA polypeptide expressed is in a disulfide-bonded dimeric form.

76. A PSMA polypeptide produced by the method of claim 71.

77. A composition, comprising the PSMA polypeptide of claim 76.

78. A method of producing a PSMA polypeptide, comprising:
transfecting cells with a vector encoding the PSMA polypeptide, and
contacting the cells with media comprising an anti-apoptotic agent, polyethylene glycol (PEG) or both.

79. The method of claim 78, wherein the anti-apoptotic agent is dextran sulfate, tropolone, a caspase inhibitor or the BCL2 gene product.

80-82. (canceled)

83. The method of claim 78, wherein the PSMA polypeptide has a cysteine-modification.

84. The method of claim 78, wherein the method further comprises harvesting and purifying the PSMA polypeptide expressed by the transfected cells.

85. The method of claim 78, wherein PSMA polypeptide expressed by the transfected cells is in a disulfide-bonded dimeric form.

86. A PSMA polypeptide produced by the method of claim 78.

87. A composition, comprising the PSMA polypeptide of claim 86.
